(19) United States
(12) Patent Application Publication
(10) Pub. No.: US 2026/0183363 A1
(43) Pub. Date: Jul. 2, 2026

(54) INHIBITION OF LUTEINIZING HORMONE (LH) ACTION AS TREATMENT OF PERIMENOPAUSAL AND POSTMENOPAUSAL SYMPTOMS AND COMPLICATIONS

(71) Applicant: RIGSHOSPITALET, Copenhagen Ø (DK)

(72) Inventor: Martin Blomberg JENSEN, Charlottenlund (DK)

(73) Assignee: RIGSHOSPITALET, Copenhagen Ø (DK)

(21) Appl. No.: 19/116,577

(22) PCT Filed: Sep. 29, 2023
(86) PCT No.: PCT/EP2023/077128

§ 371 Date: Mar. 28, 2025

(30) Foreign Application Priority Data
Sep. 29, 2022 (DK) PA202270470

(51) Int. Cl. A61K 38/09; A61K 38/17; A61K 39/00; A61P 3/04; A61P 15/12; C07K 16/26; C07K 16/28

(52) U.S. Cl. CPC A61K 38/09; A61K 38/1796; A61P 3/04; A61P 15/12; C07K 16/26; C07K 16/2869; A61K 2039/505; C07K 2317/34; C07K 2317/565; C07K 2317/76

(57) ABSTRACT

The present invention is based on the realization and verification that inhibition of Luteinizing hormone (LH) and/or luteinizing hormone/choriogonadotropin receptor (LHCGR) is useful in the treatment, alleviation or prevention of different medical states related to perimenopause and menopause in women or hypogonadism in men. Thus, an aspect of the present invention relates to LH and/or LHCGR inhibitors for use in the treatment of postmenopausal osteoporosis in a female subject. Another aspect relates to LH and/or LHCGR inhibitors for use in the reduction of fat mass in a postmenopausal female subject. A further aspect relates to LH and/or LHCGR inhibitors for use in the treatment, hypergonadotropic hypogonadism in a male subject. In addition, another aspect relates to LH and/or LHCGR inhibitors for use in the treatment of symptoms of perimenopause and/or postmenopause such as hot flashes, night sweats, osteoporosis, weight gain, hypothyroidism, conversion of T4 to T3, and/or primary hyperparathyroidism.

### TECHNICAL FIELD OF THE INVENTION

The present invention is based on the realization and verification that inhibition of luteinizing hormone (LH) and/or luteinizing hormone/choriogonadotropin receptor (LHCGR) is useful in the treatment, alleviation or prevention of different medical states related to perimenopause, menopause and postmenopausal symptoms and complications in women or testicular hypogonadism in men.

Thus, aspects of the present invention relate to an antibody and/or a small molecule
targeting luteinizing hormone (LH) and/or luteinizing hormone/choriogonadotropin receptor (LHCGR); and/or
being a GnRH agonist or GnRH antagonist;
   for use in the treatment, alleviation and/or prevention of osteoporosis in a perimenopausal or postmenopausal female subject; for use in the reduction of fat mass and/or prevention of increase in fat mass in a perimenopausal, menopausal and/or postmenopausal female subject; for use in the treatment, alleviation and/or prevention of hypergonadotropic hypogonadism in a male subject; and for use in the treatment, alleviation and/or prevention of symptoms/manifestations of perimenopause, menopause and/or postmenopause such as hot flashes, night sweats, osteoporosis, weight gain, hypothyroidism, conversion of T4 to T3, and/or primary hyperparathyroidism.

### BACKGROUND OF THE INVENTION

Among the US adult population aged 50 years and older, 54% have osteoporosis or low bone mass and the frequency of obesity is even worse. Osteoporosis impairs life quality, is a huge economic cost for society, and vertebral and hip fractures are associated with increased mortality. Osteoporosis is characterized by impaired bone formation relative to bone resorption resulting in loss of bone mass and susceptibility to fractures (Eastell et al. 2016). Hip fractures are largely responsible for the increased mortality associated with osteoporosis and 20% of women with osteoporotic hip fractures die within 1 year of their fracture.

Antiresorptive treatments such as bisphosphonates and denosumab and the newer anabolic treatments abaloparatide and romosozumab are effective but are not routinely used in younger patients due to lack of evidence of reduced fracture risk. Therefore, no effective and mild treatment can be used before the use of for instance bisphosphonates despite that strategies to prevent osteoporosis could save up to 50% of all hip fractures (Odén et al. 2013, Eastell et al. 2016). Serum calcium levels are maintained within a narrow range (1.18-1.32 mmol/L, ionized calcium) throughout life in both sexes by rapid-acting and potent regulators. The most potent regulators are vitamin D, parathyroid hormone (PTH), and fibroblast growth factor 23 (FGF23) that quickly in response to changes in serum calcium adjust intestinal absorption, renal excretion, and calcium mobilization from the skeletal compartment where 99% of all calcium is stored. Hyper- or hypocalcemia is therefore a relatively late pathological finding since the regulators finely balance intestinal calcium mobilization, urinary calcium excretion, and bone resorption to maintain serum calcium concentration completely stable as calcium influences the function of most organs. The active form of calcium is the ionized form that influences organ functions including heart contractility and rhythm and is buffered by albumin in serum and therefore dependent on albumin availability and pH-mediated changes in the strength of albumin.

Low levels of mainly estrogens during menopause are a major driver for developing osteoporosis (Eastell et al. 2016). However, a decline in bone density during the perimenopausal transition frequently occurs despite unchanged circulating estrogen levels, but with accompanied increase in gonadotrophins luteinizing hormone (LH) and follicle-stimulating hormone (FSH) (Ebeling et al. 1996). LH and human chorionic gonadotropin (hCG) are produced by the pituitary gland and placenta, respectively, and both bind to and activate the shared receptor luteinizing hormone/choriogonadotropin receptor (LHCGR) in humans and other mammals. LHCGR is mainly expressed in the ovary and testis but also in other organs

Animal and human studies have suggested that sex steroids also exert indirect calciotropic actions by regulating activation of vitamin D, bone resorption and intestinal calcium absorption as indicated by children born with inactivating mutations in the vitamin D receptor (VDR) who do not require calcium supplements after puberty. Newer studies have also shown steroidogenic effects on renal calcium handling that may be linked to the observed hypercalciuria in pregnant women which is a paradoxical observation due to the increased calcium demands from the fetus and higher levels of 1,25(OH)2D3 in pregnant women (Gertner, J. M. et al. 1986, Payne, R. B. et al. 1990, Cross, N. A. et al. 1995). This increased calcium excretion occurs already early in pregnancy where the level of hCG rapidly arises. Co-existence does not imply causality but if hCG mediates the effect then it may be induced by LHCGR. If high circulating levels of LH or hCG influence calcium excretion, then it may also be relevant in postmenopausal women or men with hypergonadotropic hypogonadism. Highly debated studies have suggested that FSH rather than LH influence bone health and resorption and maybe thereby calcium homeostasis, but very limited studies have investigated the influence of LHCGR activation on calcium homeostasis (Sun, L. et al. 2006, Liu, P. et al. 2017).

Menopause and loss of normal circulating sex steroids are not just inducing bone loss but impairing women health for many in several years and inducing a postmenopausal weight gain. The most prevalent symptoms are hot flashes, vaginal dryness, night sweats but also metabolic changes and an increased risk for endocrine diseases such as primary hyperparathyroidism and hypothyroidism. Circulating LH levels become elevated in response to ovarian failure and during the late perimenopause, a period characterized by relatively stable estrogen and rising LH and FSH levels and bone loss occurs more rapidly. The perimenopause starts as early as 10 years prior to menopause, which is defined as the time for the last menstruation. There is also a sharp increase in visceral adiposity during peri- and menopause (Greendale et al. 2019). Gonadectomy (high LH and high FSH) induces gain in body weight and fat mass. Moreover, hCG levels increase dramatically during the first two months of pregnancy, which is also a period with accumulation of fat mass in order to cover the energy demands of both the fetus and the mother (Sidebottom et al. 2001). Some of the effects on adipose tissue have been ascribed to changes in progesterone and estrogen levels (Lacasa et al. 2001; Gonzalez et al. 2002), but the role of LH or hCG on adipose tissue has not been described. Most of the symptoms related to peri- and menopause (hot flashes, night sweats, and metabolic changes) and complications (osteoporosis, weight gain and increased risk for primary hyperparathyroidism and hypothyroidism including demand for T3) have been attributed to the low estrogen level and can be alleviated to some extend with hormonal replacement therapy (HRT) in the form of estrogen and gestagen. HRT has some serious adverse effects such as increased breast cancer risk and risk of tromboembolism. Hence, a novel treatment that could be used to treat these symptoms and complications would be advantageous, and in particular a more efficient and/or reliable treatment with less serious adverse effects would be advantageous.

### SUMMARY OF THE INVENTION

The present invention is based on the realization and verification that inhibition of Luteinizing hormone (LH) and/or luteinizing hormone/choriogonadotropin receptor (LHCGR) is useful in the treatment, alleviation or prevention of different medical states related to perimenopause and menopause in women or hypogonadism in men. High circulating levels of LH induces calcium excretion and a secondary PTH increase will induce bone resorption which could result in osteoporosis. In addition, high LH and hCG may increase obesity directly by suppressing heat generation, thyroid hormone conversion and brown adipocyte activation leading to less brown adipocyte mass because the activity drops. Instead of heat generating cells, white adipocytes that can store energy will be generated. High LH levels are thereby possibly responsible for fat accumulation and redistribution in peri- and postmenopausal women, pregnant women, and in men with increased LH levels. Thus, a therapy blocking LH action in circumstances with high circulating LH may decrease calcium excretion and thereby decrease PTH-mediated bone resorption. As shown in example 1, LH induces calcium excretion and a secondary increase in PTH; however, since hCG exhibits the same effects on the calcium homeostasis as demonstrated in example 2, an antibody targeting their shared receptor, luteinizing hormone/choriogonadotropin receptor (LHCGR), would also be beneficial in the treatment, alleviation and/or prevention of osteoporosis. Thus, an aspect of the present invention relates to an antibody targeting luteinizing hormone (LH) and/or luteinizing hormone/choriogonadotropin receptor (LHCGR) for use in the treatment, alleviation and/or prevention of osteoporosis in a perimenopausal or postmenopausal female subject. GnRH agonist treatment leads to a short induction followed by a long suppression of the production of gonadotropins (FSH and LH). The induced persistent suppression of LH by GnRH treatment will therefore lead to lack of LH action as LH production and release from the pituitary is inhibited. This implies that GnRH agonists may act like LHCGR or LH inhibiting antibodies as they also block LH activity. GnRH agonists may in addition have other adverse effects as they in addition to blocking actions of LH also block FSH action and exert effects through GnRH receptors in other tissues.

Further, LH is produced by the gonadotropic cells in the anterior part of the pituitary gland and production and release is critically dependent on a pulsatile release of GnRH from the hypothalamus that stimulate the pituitary release of LH. GnRH agonists and antagonists may both be synthetic analogs of the GnRH peptide hormone, and achieve castrate testosterone levels by shutting down the GnRH-mediated release of luteinizing hormone (LH) and follicle-stimulating hormone (FSH) from the anterior pituitary. Both agonist and antagonist ensures a constant binding and thereby prevents the pulsatile GnRH surge that is required for the LH release. Therefore, without being bound by theory, the effect will be the same of both GnRH agonist and antagonist on serum levels of LH but not on GnRH receptors in other parts of the body.

Therefore, a GnRH agonist or GnRH antagonist will also be able to suppress LH levels and thereby induce a similar effect on hot flashes, osteoporosis and adipocyte function as an LH or LHCGR blocking antibody.

As demonstrated in example 5 and 6, LHCGR is highly expressed in mature adipocytes, and high LH and hCG may increase obesity directly by suppressing heat generation through UCP1, thyroid hormone conversion into T3 and brown adipocyte activation leading to less brown adipocytes. Instead of heat generating cells, white adipocytes that can store energy, will be generated. Hence, another aspect of the present invention relates to an antibody targeting luteinizing hormone (LH) and/or luteinizing hormone/choriogonadotropin receptor (LHCGR) for use in the reduction of fat mass and/or prevention of increase in fat mass in a perimenopausal, menopausal and/or postmenopausal female subject. Moreover, LH and hCG negatively influence mitochondrial function, which will promote aging and inhibition of LH, hCG or LHCGR will reduce aging and mitochondrial aging related diseases such as dementia.

As shown in example 3 high LH with no increase in sex steroids as you experience during menopause or gonadal failure aggravates the loss of calcium in the urine and thereby increases the risk for osteoporosis. Men or women with gonadal impairment cannot compensate with increasing sex steroids required to rescue the calciuric effect of LH surge, which may be critical in men with primary hypogonadism (also known as hypergonatropic or testicular hypogonadism). Thus, yet another aspect of the present invention relates to an antibody targeting LH and/or luteinizing hormone/choriogonadotropin receptor (LHCGR) for use in the treatment, alleviation and/or prevention of hypergonadotropic hypogonadism in a male subject.

In addition, an aspect of the present invention relates to an antibody targeting LH and/or luteinizing hormone/choriogonadotropin receptor (LHCGR) for use in the treatment, alleviation and/or prevention of symptoms of perimenopause, menopause and/or postmenopause such as hot flashes, night sweats, osteoporosis, weight gain, hypothyroidism, conversion of T4 to T3, and/or primary hyperparathyroidism. The involvement of LH and/or LHCGR in osteoporosis, obesity and the conversion of T4 to T3 have been demonstrated in example 1-2 and 5-7. Thus, an antibody targeting LH and/or LHCGR would most likely have an effect on several symptoms of perimenopause and/or menopause and improve life quality in postmenopausal patients that have a high prevalence of hypothyroidism, low conversion of T4 to T3 and primary hyperparathyroidism where LH inhibiting therapy will improve these endocrine diseases. This suggestion is supported by example 8 showing that injection of inhibiting antibodies targeting LH-β or LHCGR will lower thyroid volume which is the expected outcome due to increased T3 levels and lower TSH. Moreover, example 8 show that injection of inhibiting antibodies targeting LH-β or LHCGR will decrease both subcutaneous and visceral fat mass and increase core temperature and thus reduce the occurrence of obesity and hot flushes. Moreover, adipocytes exposed to LH has a lower expression of DIO2 that converts T4 to T3 so LH directly influence enzyme abundancy and activity.

Example 9 shows that white and brown cell adipocyte function may be improved by treatment with an LH or LHCGR blocking antibody.

Example 10 concludes that blocking circulating high levels of LH in menopause diminishes urinary calcium loss, which will reduce serum PTH and thereby also bone resorption and the risk of osteoporosis. The same effect is shown to be obtainable in male mice also.

In an aspect, the invention relates to antibody targeting LH and/or luteinizing hormone/choriogonadotropin receptor (LHCGR) for use in avoiding premature ovulation during assisted reproductive techniques such as IVF.

As also described in the example section, data supporting the invention includes:
Example 1 shows that high circulating levels of LH induces calcium excretion and the secondary PTH increase will induce bone resorption. Thus, a therapy blocking LH action in circumstances with high circulating LH may decrease calcium excretion and thereby decrease PTH-mediated bone resorption.
Example 2 shows that hCG also induce calcium excretion and increase serum PTH levels as was observed for LH in example 1. Thus, since both LH and hCG affect the calcium homeostasis in a similar way, it indicates that it is the activation of their shared receptor; LHCGR rather than the ligands themselves that is important for the calcium homeostasis effect.
Example 3 shows that high circulating LH prior to injection of hCG is important for the effect of hCG injection on calcium excretion. This indicates that patients with high LH respond in a similar way with increasing calcium excretion and high serum PTH leading to high bone resorption. The organ-specific effects may be direct or indirect but the lower the testosterone change the larger effect on calcium homeostasis was observed. This indicates that high LH with no increase in sex steroids as you experience during menopause or gonadal failure aggravates the loss of calcium in the urine and thereby increases the risk for osteoporosis. Men or women with gonadal impairment cannot compensate with increasing sex steroids required to rescue the calciuric effect of LH surge, which may be critical in men with primary hypogonadism or after hemi-orchiectomy and postmenopausal osteoporosis. Hence, a therapy blocking LH action may decrease calcium excretion and thereby decrease PTH mediated bone resorption and be a treatment option for men with increased LH levels, postmenopausal osteoporosis and symptoms related to perimenopause and postmenopause induced by the increased serum LH levels.
Example 4 shows that LHCGR is highly expressed in the kidney which indicates that LH and hCG can induce direct renal effects. The suggested influence on calcium excretion seems to not be mediated by transcriptional regulation of the main calcium transporter (encoded by TRPV5) but may be through translation or protein abundance in the membrane.
Examples 5 shows that LHCGR is highly expressed in mature adipocytes and high LH may increase obesity directly by suppressing heat generation through UCP1 and thyroid hormone conversion into T3 that will increase obesity. Hence, it is believed that LH and hCG reduce brown fat mass and thermogenesis and thyroid hormone conversion resulting in increased white cell adiposity. In sum, the data presented here indicates that white cell adiposity may be improved by treatment with an antibody blocking LH.
Example 6 shows that high LH and hCG may increase obesity directly by suppressing heat generation, thyroid hormone conversion and brown adipocyte activation leading to less brown adipocytes because the activity drops. Instead of heat generating cells, white adipocytes that can store energy will be generated. Thus, white cell adiposity may be improved by treatment with an LH antibody.
Example 7 present data indicating that a therapy blocking LH action will alleviate suppressive function of pituitary function (ACTH, cortisol, and TSH) reduce hair growth in genital, stomach and face of postmenopausal women and improve insulin sensitivity and not least hypothyroidism in patients not adequately treated with eltroxin (T4) but often requesting treatment with the active form of thyroid hormone T3 because high LH suppress conversion of T4 into T3. Particularly in perimenopausal, menopausal and postmenopausal women that have high LH may have beneficial effects of a treatment blocking LH action.
Example 8 demonstrates that blocking circulating high levels of LH in menopause diminishes weight gain, visceral and subcutaneous fat accumulation, lowers thyroid goiter (irregular growth of the thyroid) by improving thyroid function and increases core and skin temperature that will reduce the incidence and severity of hot flashes.
Example 9 presents data indicating that white and brown cell adipocyte function may be improved by treatment with an LH or LHCGR blocking antibody.
Example 10 concludes that blocking circulating high levels of LH in menopause diminishes urinary calcium loss, which will reduce bone resorption and the risk of osteoporosis.

### BRIEF DESCRIPTION OF THE FIGURES

FIG. 1 shows wild type mice treated for 10 days with either vehicle, cinacalcet, cinacalcet and LH (cina+LH), or LH. A: urinary excretion of calcium. B: urinary excretion of creatinine. C: Serum PTH. D: Body weight E: kidney weight (normalized to body weight of each mouse). F: femur weight (normalized to body weight of each mouse). All p-values were calculated using ANOVA with Dunnett's correction for multiple comparison. Data are presented individually and bars shows mean with SD.

FIG. 2 shows injection of hCG in 11 healthy men. Fasting Blood and urine sampling at baseline, 8 (not fasting), 24, 72 and 120 hours are shown. CTX and P1NP are only measured in fasting serum samples. Values are calculated as fold-change (relative to baseline) and shown as mean with SD. All p-values are calculated using ANOVA with Dunnett's correction for multiple testing. A: reproductive hormones with serum testosterone, estradiol, FSH and LH. B: calcium excretion in urine. C: serum ionized calcium, total calcium, albumin corrected calcium and albumin. D: PTH, calcitonin, CTX1 and PINP. E: active 1,25 Vitamin D, 25-OHD vitamin D, serum phosphate, urinary phosphate excretion.

FIG. 3 shows QTc interval measured by electrocardiogram in 10 healthy men at baseline and after 8 and 24 hours after injection with 5000 IU hCG.

FIG. 4 shows injection of hCG in a man with Familial Hypocalciuric Hypercalcemia (FHH, black line), compared with 11 healthy controls (grey line, mean with SD). Serum- and urine samples were drawn at baseline, 24 hours and 72 hours, additional urine sample after 2 hours. All samples are fasting values. All delta-values are calculated as fold-change (relative to baseline). A: urinary calcium excretion. B: ionized calcium. C: total calcium. D: PTH. E: albumin. F: serum change in phosphate. G: change in serum CTX1. H: change in serum PINP. I: testosterone. J: estradiol. K: LH. L: FSH.

FIG. 5 shows injection of hCG in 10 men with previous testicular cancer and subsequent orchiectomy and/or testicular radiation. Blood and urine sampling at baseline, 8, 24, 72 and 120 hours. All, but at the 8 hour time point, were fasting samples. Values are calculated as fold-change (relative to baseline). Solid lines show mean with SD whereas broken lines show each individual sample. All p-values are calculated using ANOVA with Dunnett's correction for multiple testing. Men were stratified in two groups based on baseline LH level: normal/low LH (L-LH, Black, n4) vs. elevated (>8.6 IU/L, E-LH, Grey, n6). A: ionized calcium. B: total calcium. C: PTH.

FIG. 6 shows testosterone, PTH and calcium in 356 men with testicular disease evaluated at baseline and 72 hours after injection of hCG. Both absolute values and fold-changes relative to baseline (Δ) are shown.

A-D: men stratified in two groups based on baseline PTH (elevated >6.9 pmol/L). P-values calculated in paired t-test (A) or student t-test (B). Elevated= “e”, normal=“n”.

A: PTH. B: Δ-PTH. C: men with insufficient increase in testosterone, below 6 nmol/L, decrease in total calcium. D: men with insufficient increase in testosterone, below 6 nmol/L, decrease in albumin corrected calcium.

FIG. 7 shows human adult kidney tissue cultured in an ex vivo model for 3 or 24 hours, treated with either vehicle, PTH, LH, hCG, estradiol or testosterone. A: expression of LHCGR exon 11 after 3 hours' culture, suppressed below limit of detection in 3 out of 10 vehicle treated samples and below limit of detection in 5 out of 5 testosterone treated samples. B: Expression of LHCGR exon 2-4 after three hours of culture. C: Expression of TRPV5 after 3 hours of culture. D: expression of TRPV5 after 24 hours of culture. P-values in bold were calculated using ANOVA with Dunnett's correction for multiple comparison. Data presented individually and the bars show mean with SD.

FIG. 8 shows A) induction of mRNA expression of LHCGR and ADIPOQ in testis and preadipocytes and mature adipocytes of three different cell lines. The cell lines are telomerase reverse transcriptase (TERT), multipotent adipose-derived stem (MADS), and Simpson-Golabi-Behmel syndrome (SGBS). Relative mRNA levels of LHCGR and ADIPOQ in three human cell lines of preadipocytes (day 0) and mature adipocytes (day 12) is shown. ADIPOQ is included as a positive control of white adipocyte differentiation. Expression levels are normalized to the house keeping gene TATA-box-binding protein (TBP). Data are presented as mean+SEM. N.A.: not applicable. B) Effects on UCP1 and DIO2 expression following LH and hCG treatment compared with vehicle in TERT cells.

FIG. 9 shows that LH and hCG diminish brown adipose tissue in wild-type mice. (A) Male mice were treated for 10 days with either vehicle (NaCl 0.9%×1 daily s.c.), LH (66.7 IU/kg×1 daily s.c.) or (B) vehicle or hCG (666.7 IE/kg every other day i.p.). All P-values were calculated using Student's t-test. Data points are presented individually and the bars show mean+/−SEM.

FIG. 10 shows injection of hCG in 11 healthy men. Fasting blood and urine sampling at baseline, 8 (not fasting), 24, 72 and 120 hours are shown. Values are shown as mean. All p-values are calculated using ANOVA with Dunnett's correction for multiple testing. A: Insulin, IGF1, IGFBp3 B: aldosterone, cortisol, ACTH C: serum androstenedione and 17 hydroxyprogesterone. D: TSH, T4, and T3.

FIG. 11 shows that antibodies targeting LH- or LHCGR influence body weight, visceral and adipose tissue, and thyroid mass in a model of menopause (female gonadectomy model). Female mice with no ovaries were treated for 10 days with either vehicle (n: 6) or Antibodies against LH or LHCGR or a GnRH antagonist (n: 3 for each group). The figures shows (A) changes in total body weight (BW); (B) changes in subcutaneous (sub cut), visceral adipose tissue or thyroid weight; and (C) relative changes in body weight during the intervention following treatment for 10 days. Data are presented as means.

FIG. 12 shows that antibodies targeting LH-β or LHCGR influence temperature regulation in a model of menopause (female gonadectomy model). Female mice with no ovaries were treated for 10 days with either vehicle (n: 6) or antibodies against LH or LHCGR or a GnRH antagonist (n: 3 for each group). The figures shows (A) Changes in core temperature. (B) Skin temperature at the tail and (C) Skin temperature at the head before intervention, 5, and 10 days after the intervention. Data are presented as means.

FIG. 13 shows changes in the expression of specific selected genes involved in adipocyte function and thyroid hormone conversion. (A) changes in genes (LEP, NAMPT, APOE, and ADIPOQ) important for adipocyte function and obesity. (B) Changes in the enzyme converting thyroid hormone T4 to T3 DIO2 in three different fat cell lines (2 white and 1 brown) treated with vehicle, LH, hCG alone or in combination with sex steroids.

FIG. 14 shows that antibodies targeting LH-β or LHCGR and a GnrH antagonist influence calcium excretion and kidney size a model of menopause (female gonadectomy model) and normal male mice. All mice were treated for 10 days with either vehicle (n: 6) or Antibodies against LH or LHCGR or a GnRH antagonist (only female) (A) Changes in kidney weight. (B) Changes in urinary calcium concentration in both female mice. (C) Changes in urinary calcium concentration in male mice. 10 days after the intervention. Data are presented as means.

The present invention will now be described in more detail in the following.

### DETAILED DESCRIPTION OF THE INVENTION

### Definitions

Prior to discussing the present invention in further details, the following terms and conventions will first be defined:

### Luteinizing Hormone (LH)

Luteinizing hormone (LH), also known as “luteinising hormone”, “lutropin” and “lutrophin” is a hormone produced by gonadotropic cells in the anterior pituitary gland. LH is a heterodimeric glycoprotein. Each monomeric unit is a glycoprotein molecule; one alpha and one B-subunit make the full, functional protein. The alpha subunits of LH, FSH, TSH, and hCG are identical, whereas the B-subunits vary. LH has a-subunit of 120 amino acids (LHB) that confers its specific biologic action and is responsible for the specificity of the interaction with the LH receptor. This B-subunit contains an amino acid sequence that exhibits large homologies with that of the B-subunit of hCG and both stimulate the same receptor. However, the hCG B-subunit contains an additional 24 amino acids, and the two hormones differ in the composition of their sugar moieties.

### Luteinizing Hormone/Choriogonadotropin Receptor (LHR)

The “luteinizing hormone/choriogonadotropin receptor” (LHCGR), also known as “lutropin/choriogonadotropin receptor” (LCGR), “LH/CGR” or simply “luteinizing hormone receptor” (LHR) is a transmembrane receptor found predominantly in the ovary and testis, but also many extragonadal organs such as the uterus and breasts. The receptor consists of 11 exons and there exist multiple isoforms and the receptor can also be found in circulation.

The receptor interacts with both luteinizing hormone (LH) and chorionic gonadotropins (such as hCG in humans).

### Gonadotropin-Releasing Hormone (GnRH)

Gonadotropin-releasing hormone (GnRH) is a releasing hormone responsible for the release of follicle-stimulating hormone (FSH) and luteinizing hormone (LH) from the anterior pituitary. GnRH is a tropic peptide hormone synthesized and released from GnRH neurons within the hypothalamus. The peptide belongs to gonadotropin-releasing hormone family. It constitutes the initial step in the hypothalamic-pituitary-gonadal axis.

In the present context, the terms “GnRH agonist” and “GnRH antagonist” refer to compounds, which either promote or inhibit GnRH action through GnRH-receptors respectively. Both types of compounds impair oulsatile activation of GnRH receptors and thereby blocks release of LH and FSH.

### Postmenopausal Woman

In the present context, a “postmenopausal woman” is defined as a woman who has gone through the menopause. Postmenopause is defined as the menstrual period has been gone for longer than 12 consecutive months.

### Menopause

Is determined retrospectively as the time of last menstruation.

### Perimenopause

The 10 year time-period before menopause wherein women start to experience symptoms of menopause but still have menstruation. During this phase, serum LH and FSH starts to increase and the pulsatility of estrogen declines although serum estradiol are often normal.

### Hypergonadotropic Hypogonadism (HH)

“Hypergonadotropic hypogonadism” (HH), also known as “primary or peripheral/gonadal hypogonadism”, is a condition which is characterized by hypogonadism due to an impaired response of the gonads to the gonadotropins, follicle-stimulating hormone (FSH) and luteinizing hormone (LH), and in turn a lack of sex steroid production and elevated gonadotropin levels.

### White Adipose Tissue/White Fat

“White adipose tissue” or “white fat” is one of the two types of adipose tissue found in mammals. There exist different types of white adipocytes, which is important for storage but also endocrine functions and thermogenesis. In humans, the healthy amount of white adipose tissue varies with age, but composes between 6-25% of body weight in adult men and 14-35% in adult women.

### Brown Adipose Tissue/Brown Fat

“Brown adipose tissue” or “brown fat” makes up the adipose organ together with white adipose tissue. Brown adipose tissue is found in almost all mammals. Brown fat mass declines after menopause and is important for generating heat and core temperature.

### Osteoporosis

Osteoporosis is a systemic skeletal disorder characterized by low bone mass, micro-architectural deterioration of bone tissue leading to bone fragility, and consequent increase in fracture risk.

### Primary Hyperparathyroidism

Is a systemic disorder characterized by an adenoma in the parathyroid gland that produces and releases high circulating PTH that increases serum calcium and induces hypercalcemia due to increased bone resorption and increased activation of vitamin D and consequent increase in fracture risk and increased mortality. This disease in 2-3-fold more prevalent in postmenopausal women. The disease is characterized by a high normal or elevated PTH level and hypercalcemia and hypercalciuria.

### Hypothyroidism

Is a systemic disorder often characterized by immunological degradation of the thyroid gland, which is more prevalent in postmenopausal women. Almost all women with hypothyroidism are treated with T4 treatment but a fraction are not satisfied and require treatment with T3, which is the active form of thyroid hormones that normally is formed by deiodination in the fat cells by the enzyme DIO2.

### Pharmaceutical Acceptable

In the present context, the term “pharmaceutically acceptable” refers to molecular entities, compositions and methods that are suitable for use with humans and/or animals without undue adverse side effects (such as toxicity, irritation and allergic response) commensurate with a reasonable benefit/risk ratio.

### Excipient

In the present context, the term “excipient” refers to a natural or synthetic substance formulated alongside the active or therapeutic ingredient (an ingredient that is not the active ingredient) of a medication, included for the purpose of stabilization, bulking, or to confer a therapeutic enhancement on the active ingredient in the final dosage form, such as facilitating drug absorption, reducing viscosity, enhancing solubility, adjusting tonicity, mitigating injection site discomfort, depressing the freezing point, or enhancing stability. The term may refer to a diluent, adjuvant, carrier, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### Carrier

In the present context, the term “carrier” refers to any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

### Small Molecule

A small molecule is a low molecular weight (≤1000 Daltons) organic compound that may regulate a biological process. Larger structures such as nucleic acids and proteins, and many polysaccharides are not small molecules, although their constituent monomers (ribo- or deoxyribonucleotides, amino acids, and monosaccharides, respectively) are often considered small molecules.

Medical uses of antibodies and/or small molecules being a GnRH agonist or targeting luteinizing hormone (LH) and/or luteinizing hormone/choriogonadotropin receptor (LHCGR)

### Treatment of Osteoporosis

The present invention demonstrates that high circulating levels of LH induces calcium excretion and a secondary PTH increase will induce bone resorption. Hence, a therapy blocking LH action in circumstances with high circulating LH may decrease calcium excretion and thereby decrease PTH-mediated bone resorption. As shown in example 1, LH induces calcium excretion and a secondary increase in PTH; however, since hCG exhibits the same effects on the calcium homeostasis as demonstrated in example 2, an antibody targeting their shared receptor, luteinizing hormone/choriogonadotropin receptor (LHCGR), would also be beneficial in the treatment, alleviation and/or prevention of osteoporosis. Thus, an aspect of the present invention relates to an antibody and/or a small molecule
targeting luteinizing hormone (LH) and/or luteinizing hormone/choriogonadotropin receptor (LHCGR); and/or
being a GnRH agonist or GnRH antagonist;
   for use in the treatment, alleviation and/or prevention of osteoporosis in a perimenopausal or postmenopausal female subject.

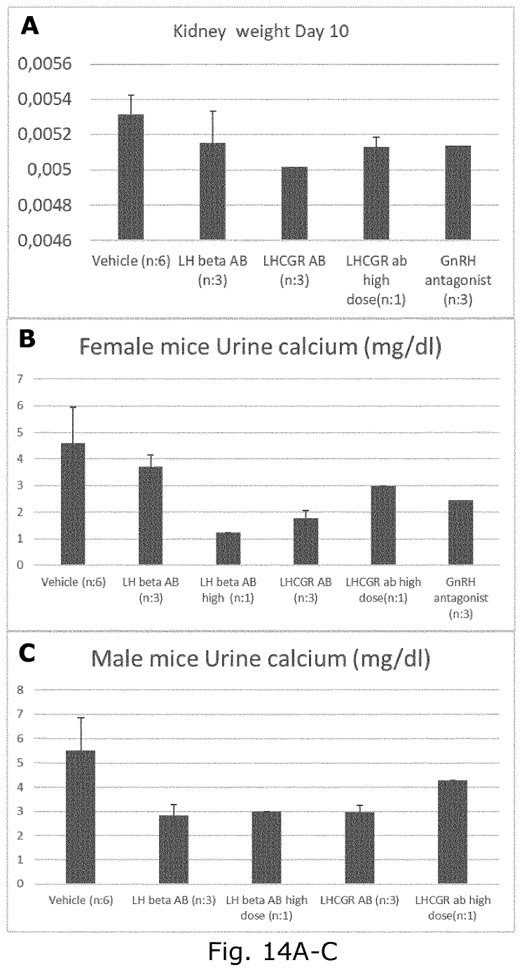

In a similar aspect, the present invention relates to an antibody and/or a small molecule
targeting luteinizing hormone (LH) and/or luteinizing hormone/choriogonadotropin receptor (LHCGR); and/or
being a GnRH agonist or GnRH antagonist;
   for use in the treatment, alleviation and/or prevention of osteoporosis in a male subject. As seen in example 10 (and FIG. 14C), Urine calcium is also reduced in male mice after treatment according to the invention.

### Treatment of Increased Fat Mass

As demonstrated in example 5 and 6, LHCGR is highly expressed in mature adipocytes, and high LH and hCG may increase obesity directly by suppressing heat generation through UCP1, thyroid hormone conversion into T3 and brown adipocyte activation leading to less brown adipocytes. Instead of heat generating cells, white adipocytes that can store energy, will be generated. Further, Example 8 shows that the change in body weight following treatments with antibodies targeting LH or LHCGR and GnRH antagonist were effective in suppressing the effect of menopause (gonadectomy) on weight induction.

Hence, another aspect of the present invention relates to an antibody and/or a small molecule
targeting luteinizing hormone (LH) and/or luteinizing hormone/choriogonadotropin receptor (LHCGR); and/or
being a GnRH agonist or GnRH antagonist;
   for use in the reduction of fat mass and/or prevention of increase in fat mass in a perimenopausal, menopausal and/or postmenopausal female subject.

In an embodiment, said subject has a BMI below 30, such as in the range 18.5-24.9 (considered healthy) or such as in the range 25-29.9 (considered overweight).

In an embodiment, the reduction of fat mass and/or prevention of increase in fat mass in an obese perimenopausal, menopausal and/or postmenopausal female subject.

In the present context, an obese subject is considered a subject having a BMI of or higher.

In yet a related embodiment, the invention relates to the (non-medical) use of an antibody and/or a small molecule
targeting luteinizing hormone (LH) and/or luteinizing hormone/choriogonadotropin receptor (LHCGR); and/or
being a GnRH agonist or GnRH antagonist
   for reduction of fat mass and/or prevention of increase in fat mass in a perimenopausal, menopausal and/or postmenopausal female subject.

Again, in an embodiment, the reduction of fat mass and/or prevention of increase in fat mass in an obese (BMI of 30 or higher) perimenopausal, menopausal and/or postmenopausal female subject.

In an embodiment, said subject has a BMI below 30, such as in the range 18.5-24.9 (considered healthy) or such as in the range 25-29.9 (considered overweight).

In another embodiment, said subject has a BMI in the range 18.5-24.9.

### Treatment of Hypergonadotropic Hypogonadism

As shown in example 3 high LH with no increase in sex steroids as you experience during menopause or gonadal failure aggravates the loss of calcium in the urine and thereby increases the risk for osteoporosis. Men or women with gonadal impairment cannot compensate with increasing sex steroids required to rescue the calciuric effect of LH surge, which may be critical in men with primary hypogonadism (also known as hypergonatropic hypogonadism). Thus, yet another aspect of the present invention relates to an antibody and/or a small molecule
targeting LH and/or luteinizing hormone/choriogonadotropin receptor (LHCGR); and/or
being a GnRH agonist or GnRH antagonist;
   for use in the treatment, alleviation and/or prevention of hypergonadotropic hypogonadism in a male subject, such as alone or in combination with testosterone.
   Treatment of Symptoms/Manifestations of Perimenopause and/or Postmenopause

In addition, an aspect of the present invention relates to an antibody and/or a small molecule
targeting LH and/or luteinizing hormone/choriogonadotropin receptor (LHCGR); and/or
being a GnRH agonist or GnRH antagonist;
   for use in the treatment, alleviation and/or prevention of symptoms/manifestations of perimenopause, menopause and/or postmenopause such as hot flashes, night sweats, osteoporosis, weight gain, hypothyroidism, conversion of T4 to T3, and/or primary hyperparathyroidism. The involvement of LH and/or LHCGR in osteoporosis, obesity, and the conversion of T4 to T3 have been demonstrated in example 1-2 and 5-7 and 8-10. In addition, example 5 demonstrated that LH and hCG treatment suppressed UCP1 expression which is important since UCP1 together with thyroid hormones are important for thermoregulation and hot flashes. Thus, an antibody targeting LH and/or LHCGR can have an effect on several symptoms of perimenopause and/or menopause. In addition, GnRH agonist treatment leads to a short induction followed by a long suppression of the production of gonadotropins (FSH and LH). The induced persistent suppression of LH by GnRH treatment will therefore lead to lack of LH action as LH production and release from the pituitary is inhibited. This implies that GnRH agonists may act like LHCGR or LH inhibiting antibodies as they also block LH activity. GnRH agonists may in addition have other adverse effects as they in addition to blocking actions of LH also block FSH action and exert effects through GnRH receptors in other tissues. Therefore, a GnRH agonist will induce similar actions as antibodies blocking LH action and may be used to treat postmenopausal symptoms and complications.

As outlined further above, GnRH antagonists may have the same effect on LH as GnRH agonists.

An aspect relates to an antibody and/or a small molecule
targeting LH and/or luteinizing hormone/choriogonadotropin receptor (LHCGR); and/or
being a GnRH agonist or GnRH antagonists;
   for use in the treatment, alleviation and/or prevention of endometriosis and/or Polycystic ovary syndrome (PCOS) in a female subject.

An aspect relates to an antibody and/or a small molecule
targeting LH and/or luteinizing hormone/choriogonadotropin receptor (LHCGR); and/or
being a GnRH agonist or GnRH antagonists;
   for use in the treatment, alleviation and/or prevention of myxedema and/or primary hyperparathyroidism.

In a preferred embodiment, the antibody targets luteinizing hormone (LH). In a more preferred embodiment, the antibody and/or the small molecule targets luteinizing hormone (LH), wherein said antibody binds specifically to an epitope in the β-subunit of LH.

In an embodiment, said antibody and/or small molecule binds specifically to an epitope in the β-subunit of LH, within position 21-141 of SEQ ID NO: 1.

In yet a preferred embodiment, the antibody and/or the small molecule for use is an antibody. In examples 8-10, antibodies against LH and LHCGR have been tested.

In another preferred embodiment, the antibody is an antibody targeting LH.

In yet another embodiment, the antibody is an antibody targeting LHCGR.

In an embodiment, said antibody and/or small molecule binds specifically to an epitope on LHCGR and blocks activation and/or binding of luteinizing hormone (LH) to luteinizing hormone/choriogonadotropin receptor (LHCGR).

In a further embodiment, said antibody and/or small molecule binds specifically to an epitope on LHCGR, within position 27-362 of SEQ ID NO: 2.

In another embodiment, said antibody binds specifically to an epitope in the β-subunit of LH and blocks activation and/or binding of luteinizing hormone (LH) to luteinizing hormone/choriogonadotropin receptor (LHCGR).

In yet another embodiment, the antibody is selected from the group consisting of a polyclonal antibody, a monoclonal antibody, an antibody wherein the heavy chain and the light chain are connected by a flexible linker, an Fv molecule, an antigen binding fragment, a Fab fragment, a Fab′ fragment, a F(ab′) 2 molecule, a fully human antibody, a humanized antibody, a chimeric antibody, a fragment of an antibody and a single-domain antibody (sdAb) (nanobody).

In an embodiment, the weight of the small molecule is equal to or below 1000 Dalton (Da), such as equal to or below 900 Da, such as equal to or below 500 Da.

In an embodiment, the antibody and/or the small molecule is a small molecule.

In yet an embodiment, the small molecule is a GnRH agonist.

In a more specific embodiment, the GnRH agonist is selected from the group consisting of Camcevi, Eligard, Fensolvi, Goserelin, Histrelin, Leuprolide, Lupron Lupron depot, triptorelin, histrelin, nafarelin and combinations thereof.

In an embodiment, the small molecule is selected from the group consisting of Goserelin (CAS No 65807-02-5), Leuprorelin (CAS No 53714-56-0), Triptorelin (CAS No 57773-63-4), and Nafarelin (CAS No 76932-56-4). As outlined above, these compounds are GnRH agonists.

In another embodiment, the small molecule is a GnRH antagonist.

In a more specific embodiment, the GnRH antagonist is selected from the group consisting of Antagon, Cetrorelix, Cetrotide, Elagolix, elagolix/estradiol/norethindrone acetate, Ganirelix, Orgalutran, Oriahnn and combinations thereof. In the example section (examples 8-9) the GnRH antagonist Cetrorelix (Merck) has been used.

In a preferred embodiment, the GnRH agonist or GnRH antagonist is a small molecule.

In a further embodiment, the subject has a level of:
luteinizing hormone (LH) above 10 IU/L, such as above 15 IU/L in blood serum, such as above 20 IU/L in blood serum, such as above 25 IU/L LH, preferably above 30 IU/L in blood serum, such as above 35 IU/L LH or such as above 40 IU/L LH; and/or
anti mullerian hormone (AMH) below 10 pmol/L in blood serum; and/or
Estrogen below 30 pg/mL, such as below 25 pg/mL, such as below 20 pg/mL in blood serum.

An embodiment of the present invention relates to the antibody and/or the small molecule for use in the treatment, alleviation and/or prevention of hypergonadotropic hypogonadism in a male subject, wherein said male subject has undergone hemi-orchiectomy. Some of the subjects investigated in example 3 had undergone hemi-orchiectomy and high LH is a frequent finding in the endocrine clinic in men with hypogonadism or after hemi-orchiectomy. Thus, an antibody targeting LH or LHCGR could be used to treat, alleviate or prevent weight gain and osteoporosis weight hypergonadotropic hypogonadism in men who have undergone hemi-orchiectomy.

The data presented in examples 5 and 6 indicate that white cell adiposity may be improved by treatment with an LH antibody. Hence, in an embodiment of the present invention, relating to the use in the reduction of fat mass and/or prevention of increase in fat mass in a perimenopausal, menopausal and/or postmenopausal female subject, the reduction of fat mass and/or prevention of increase in fat mass is reduction of white adipose tissue and/or prevention of increase in white adipose tissue.

In an embodiment, the reduction of fat mass and/or prevention of increase in fat mass, is reduction of visceral and/or subcutaneous fat mass and/or prevention of increase in visceral and subcutaneous fat mass. As seen in example 8, blocking LH action either by blocking LH-β or the receptor LHCGR with antibodies reduces the weight gain normally experienced following menopause. Interestingly, the lower body weight can be explained by reduced visceral fat mass for all treatments but also subcutaneous fat for LH-β antibody.

Thus, in a related embodiment, the reduction of fat mass and/or prevention of increase in fat mass, is reduction of visceral fat mass and/or prevention of increase in visceral fat mass.

In yet a related embodiment, the reduction of fat mass and/or prevention of increase in fat mass, is reduction of subcutaneous fat mass and/or prevention of increase in subcutaneous fat mass.

In addition, in an embodiment the reduction of fat mass and/or prevention of increase in fat mass is by increased thermogenesis and/or increased thyroid hormone conversion.

In an embodiment, the subject is a mammal, such as selected from the groups consisting of human, pig, cattle, zebu, donkey, horse, dog, cat, goat, and sheep, preferably a human.

In a preferred embodiment, the subject is a human. Human male subjects have been investigated in examples 2, 3, 4 and 7. In examples 8-10, mice models of e.g. menopausal human female subjects have been used.

In an embodiment, the antibody and/or the small molecule is formulated as a pharmaceutical composition.

In yet an embodiment, the pharmaceutical composition comprises one or more pharmaceutical acceptable excipients and/or carriers.

In yet another embodiment, the antibody and/or the small molecule is formulated as a nutritional formulation, such as a health supplement.

It should be noted that embodiments and features described in the context of one of the aspects of the present invention also apply to the other aspects of the invention. In particular, embodiments for one medical use may also be combined with the other listed medical uses.

### Other Aspects of the Invention

An aspect of the invention relates to a method treating, preventing or alleviating a disease or disorder in a subject in need thereof, the method comprising administering to the subject an antibody and/or a small molecule
targeting luteinizing hormone (LH) and/or luteinizing hormone/choriogonadotropin receptor (LHCGR); and/or
being a GnRH agonist or GnRH antagonist;
   wherein the treating, preventing or alleviating of disease or disorder is selected from the group consisting of:
reduction of fat mass and/or prevention of increase in fat mass in a perimenopausal, menopausal and/or postmenopausal female subject;
treatment, alleviation and/or prevention of osteoporosis in a perimenopausal or postmenopausal female subject;
treatment, alleviation and/or prevention of osteoporosis in a male subject;
treatment, alleviation and/or prevention of hypergonadotropic hypogonadism in a male subject.
treatment, alleviation and/or prevention of symptoms/manifestations of perimenopause, menopause and/or postmenopause such as hot flashes, night sweats, osteoporosis, weight gain, hypothyroidism, conversion of T4 to T3, and/or primary hyperparathyroidism;
treatment, alleviation and/or prevention of suppressed mitochondrial function;
treatment, alleviation and/or prevention of risk of age-related diseases such as metabolic syndrome, thrombosis and dementia, such as Alzheimers disease.

Yet an aspect of the invention relates to an (LH targeting) antibody comprising a variable Light chain comprising
CDR1 according to SEQ ID NO: 3;
CDR2 with the amino acid sequence KVS; and
CDR3 according to SEQ ID NO: 4;
   and
   a variable heavy comprising:
CDR1 according to SEQ ID NO: 6;
CDR2 according to SEQ ID NO: 7; and
CDR3 according to SEQ ID NO: 8;

In an embodiment, the (LH targeting) antibody comprising
a variable Light chain sequence according to SEQ ID NO: 5; and
a variable Heavy chain sequence according to SEQ ID NO: 9.

Such antibody is targeting mice LH and has been tested in examples 8-10.

In an embodiment, said antibody is a humanized antibody.

All patent and non-patent references cited in the present application, are hereby incorporated by reference in their entirety.

The invention will now be described in further details in the following non-limiting examples.

### EXAMPLES

### Example 1—Mice Treated with Luteinizing Hormone (LH)

### Aim of Study

The aim of this study was to investigate the effect of LH and LH in combination with Cinacalcet on calcium excretion in urine, the creatinine level in urine, PTH level in serum, the body weight of experimental mice, and lastly the kidney and femur weight in said mice.

### Materials and Methods

Eight weeks old male wild type mice (C57BL/6), caged at Biotest Facility, Trige, Denmark, were treated for 10 days with either:
vehicle (n=12; NaCl 0.9%×1 daily s.c.);
Cinacalcet (n=8; 30 mg/kg, ×1 daily), (Cinacalcet is in a class of medications called calcimimetics which works by signaling the body to produce less parathyroid hormone (PTH) in order to decrease the amount of calcium in the blood);
LH (n=8; 66.7 IU/kg×1 daily s.c.);
LH in combination with Cinacalcet (n=8; 66.7 IU/kg×1 daily s.c+cinacalcet orally 30 mg/kg once daily); or
hCG (n=8; 666.7 IU/kg, every other day, i.p.).

At termination, blood and urine sampling was performed, animals were sacrificed and body weight was assessed. Serum PTH was measured using commercially available ELISA kit Mouse PTH 1-84 from Immutopics. Plasma and urinary calcium and creatinine was measured using Stanbio LiquiColor (Arzenazo III) product numbers 0155 and 0430, respectively. All analyses of mice serum, urine, and organ weight were analyzed with ANOVA with control/baseline as reference value. Each p-value presented was adjusted for multiple comparisons with Dunnett's post hoc test.

### Results

### Injecting LH Changes Renal Calcium Handling in Mice

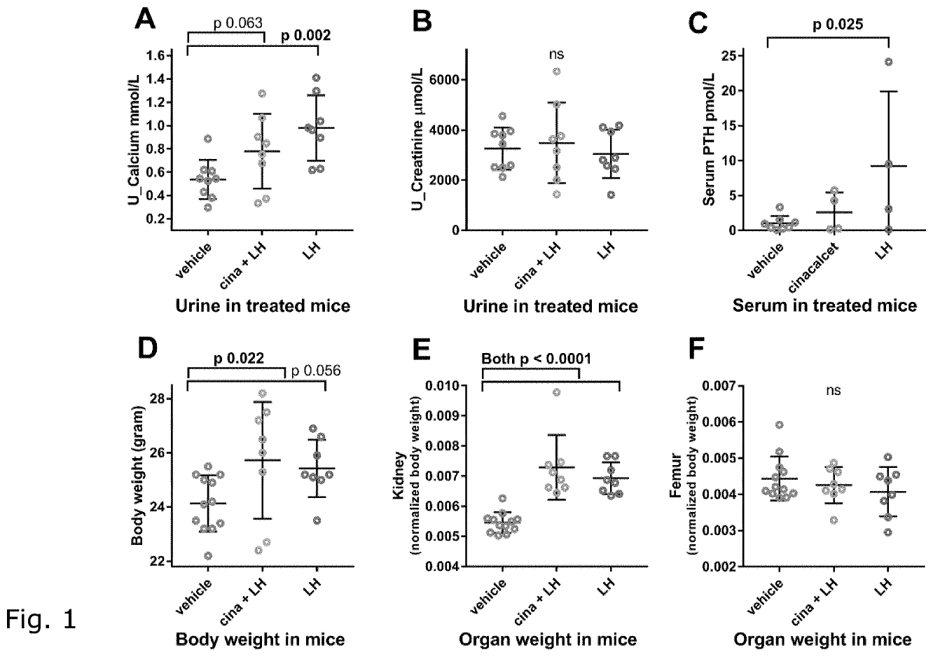

Urinary calcium excretion and calcium/creatinine ratio was significantly higher, and it almost doubled after 10 days of treatment with LH compared with vehicle-treated mice (0.98 mmol/L SD 0.28 vs. 0.54 mmol/L SD 0.17, p=0.002). The mice treated with the combined treatment of LH and cinacalcet also had increased calcium excretion (0.78 mmol/L, SD 0.32), however not reaching significance (FIG. 1A).

hCG treatment alone or in combination with any other treatment did not affect calcium excretion (data not shown). Urinary creatinine was measured to standardize calcium excretion and neither LH+cinacalcet-treated nor LH-treated mice were affected (FIG. 1B).

LH treatment increased serum PTH levels compared with vehicle-treatment (9.2 pmol/L vs. 1.0 pmol/L, p=0.025) (FIG. 1C) even though a substantial number of mice had samples below detection limit due to the use of serum rather than plasma.

Bodyweight was significantly higher in LH+cinacalcet-treated mice (p=0.018), compared with vehicle-treated mice and borderline higher in LH-treated mice (p=0.053, FIG. 1D). Despite being larger, both LH+cinacalcet-treated and LH-treated mice had significantly lower kidney size, compared with vehicle, before and after adjusting for body weight (all p<0.00001, FIG. 1E). Femoral bone mass was not different between treatment groups neither with nor without adjusting for body weight (FIG. 1F).

### Conclusion

This study suggests a basal physiological, yet undescribed endocrine crosstalk between the pituitary gland and kidney, proposing LH as a novel inducer of urinary calcium excretion and regulator of calcium homeostasis. This implies that the effect of increasing calcium excretion because of LH treatment leads to a secondary increase in PTH that will lead to increased bone resorption. This effect is not a temporary effect caused by a single injection since the observed effect here is based on a two-week treatment with LH that following the whole treatment period still induces an increased urinary calcium loss and high serum PTH. Besides increasing calcium excretion, these short-term treatments with LH induced a significant increase in kidney size that could be fully reversible.

This suggestion is in line with two different breeds of gain-of-function mice over-expressing hCG both presenting a clear phenotype with degenerating kidneys, that the authors hypothesized was secondary and not a direct effect of hCG. There was an increase in body weight in LH+cinacalcet-treated and LH-treated mice, which may be secondary to testosterone and muscle increase, as weight of femoral bones was equal in all groups. However, it is surprising that femoral mass was not increased due to the high circulating sex steroids which may imply that the calciuric effect and PTH increase is reversing the effect on the bone or that the treatment period is too short to see a significant effect.

The conclusion of the study is that circulating high levels of LH induce calcium excretion and the secondary PTH increase will induce bone resorption. A therapy blocking LH action in circumstances with high circulating LH may therefore decrease calcium excretion and thereby decrease PTH-mediated bone resorption. Post-menopausal woman has increased levels of LH. Thus, by inhibiting LH, calcium excretion can be prevented/limited and thereby prevent osteoporosis and post-menopausal osteoporosis and the risk of developing primary hyperparathyroidsism.

### Example 2−Injection of Chorionic Gonadotropin (hCG) in Healthy Men

### Aim of Study

LH and hCG works through the same receptor; luteinizing hormone/choriogonadotropin receptor (LHCGR), but hCG has a longer half-life in serum compared with LH. The aim of this study was therefore to investigate the relationship between calcium homeostasis, reproductive hormones and hCG in healthy men. To test a potential interaction, the inventors injected a large dose of hCG and thereby increased endogenous sex steroids and followed the men for subsequent changes in calcium homeostasis.

### Materials and Methods

### Cohorts:

### Clinical Intervention

11 healthy men and 10 men with previous testicular cancer and hemi-orchiectomy and/or testicular radiation were invited to participate in a prospective clinical trial, receiving 5000 IU hCG (Pregnyl) intramuscularly. They were followed longitudinally with blood- and urine sampling at baseline and 2, 8, 24, 72 and 120 hours after injection. All samples were drawn while fasting except after 8 hours. Sampling and subsequent injection at baseline and sampling at 24, 72, and 120 hours were done between 8.00 am and 9.30 am. Electro-cardiogram (ECG) was monitored at baseline and after 8 and 24 hours and QTc interval was calculated using Bazett's formula. Age, BMI and reproductive hormones on the 11 healthy men have previously been published in a baseline table. The inventors also investigated one man with an inactivating mutation in his Calcium Sensing Receptor c.2454G>A, p.Trp818 heterozygote, resulting in Familial Hypocalciuric Hypercalcemia (FHH) who was evaluated in our andrological clinic fasting blood- and urine samples which were collected at baseline, 2, 24 and 72 hours after injection of 5000 IU hCG. Serum testosterone was measured by LC-MS/MS in the 21 men included in the prospective clinical intervention study and estradiol (CV 13%) was determined by radioimmunoassay (Pantex, Santa Monica, CA). Serum FSH and LH levels were also measured with Delfia, CV<5% for both. Measurements of 25-OH vitamin D and 1,25 active vitamin D were performed by LC-MS with CV of <10% and 18%, respectively. CTX1 (CV 8%) and P1NP (CV 8%) were both measured on IDSi10. Serum and urinal total calcium (CV 2.5%), creatinine (CV 5%), and phosphate (CV 6%), and serum calcitonin (CV 7%) and serum PTH (CV<4%) were all measured using Cobas 8000 while serum ionized calcium (CV 3%) was measured on ABL 837. Longitudinal assessment of blood- and urine samples (delta values) were calculated as fold change relative to baseline for each person with all samples' initial value starting at one. All analyses were analyzed with ANOVA with control/baseline as reference value. Every p-value presented was adjusted for multiple comparisons with Dunnett's post hoc test. However, evaluation of changes in QTc interval on ECGs was available only at two time points and therefore statistically analyzed using paired T-test.

### Results

### Calcium Homeostasis in Healthy Men is Influenced by a Single Injection of hCG

Baseline characteristics including reproductive hormones and calcium homeostasis of the 11 healthy men injected with 5000 IU hCG are presented in Table 1.

TABLE 1
Age, BMI, reproductive hormones and calcium homeostasis
in 11 healthy men, and one man with familial hypocalciuric
hypercalcemia (FHH) are depicted.

| | Healthy Controls (n11) | | | | FHH | Normal | |
|---|---|---|---|---|---|---|---|
| | Mean | Median | SD | IQR | patient | Range | Unit |
| Age | 31.00 | 29.00 | 8.21 | 15.00 | 34.00 | — | years |
| BME | 23.14 | 23.46 | 2.06 | 2.53 | — | 18-25 | kg/m2 |
| Testosterone | 19.64 | 19.49 | 3.59 | 6.16 | 19.20 | 8-29* | nmol/L |
| Estradiol | 88.00 | 86.00 | 26.98 | 47.00 | 62.00 | 40-180* | pmol/L |
| LH | 3.31 | 2.67 | 1.55 | 2.60 | 3.56 | 1.7-8.6 | IU/L |
| FSH | 3.97 | 3.49 | 2.71 | 4.22 | 4.20 | 1.5-12.9 | IU/L |
| Ionized Calcium | 1.21 | 1.20 | 0.03 | 0.04 | 1.43 | 1.18-1.32 | mmol/L |
| Total Calcium | 2.41 | 2.40 | 0.07 | 0.09 | 2.67 | 2.15-2.51 | mmol/L |
| PTH | 3.75 | 3.20 | 1.67 | 1.10 | 3.10 | 1.6-6.9 | pmol/L |
| Phosphate | 1.07 | 1.01 | 0.20 | 0.34 | 1.12 | 0.71-1.53 | mmol/L |
| 1,25 DiOH D-vitamin | 88.73 | 66.00 | 52.69 | 98.00 | — | 37-216 | pmol/L |
| 25-OH D-vitamin | 51.82 | 44.00 | 34.03 | 42.00 | — | >50 | nmol/L |

All samples are drawn between 8.00-9.30 am and while fasting.
All men were subsequently injected with hCG and followed for up to 120 days.
ANOVA compares healthy controls with men with previous testis cancer.
*normal range is approximate as level varies with age.
SD: standard deviation.
IQR: interquartile range

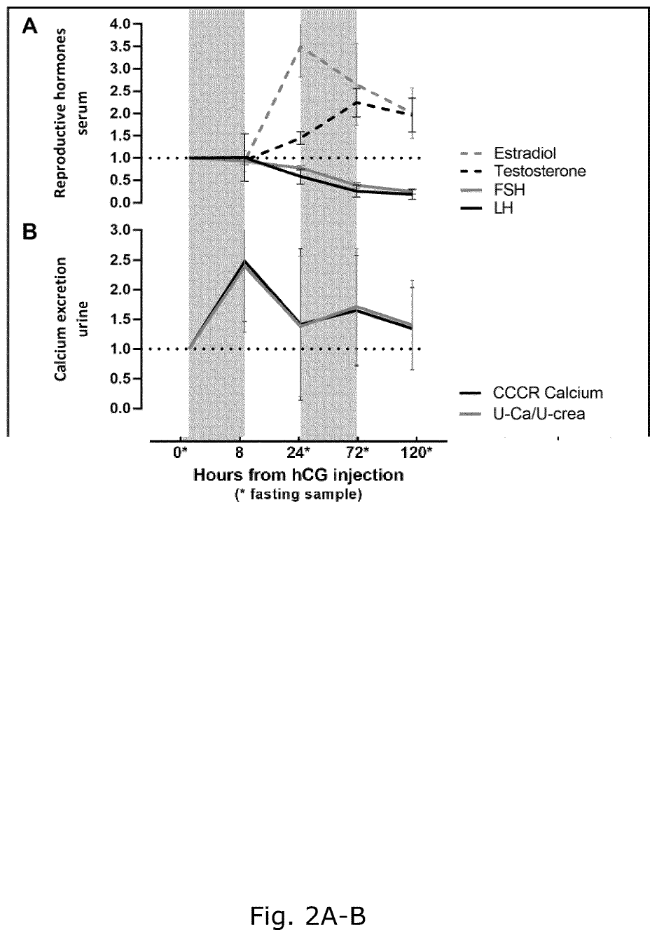

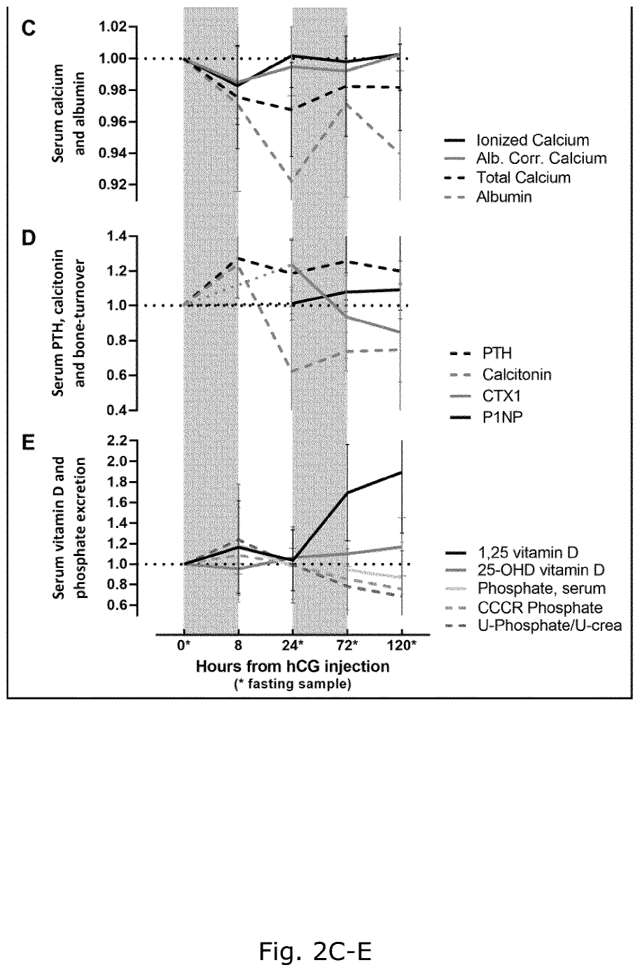

hCG injection induced an expected, significant increase in estradiol and testosterone (FIG. 2A). Noteworthy, the increase in sex steroids was not observed until after 24 hours and onwards with a peak for estradiol after 24 hours and testosterone after 72 hours (3.5 fold and 2.2 fold increase, respectively). Due to negative feedback, this was mirrored by a decrease in LH and FSH. A significant >2-fold increase in urinary calcium excretion was observed already 8 hours after injection of hCG (2.4 fold increase in U-Calcium/U-creatinine ratio: p=0.002, 2.5 fold increase in CCCR: p=0.001) (FIG. 2B). The urinary excretion remained 50% higher than baseline levels throughout the study period, though not statistically significant after correction for multiple comparisons. Calcium excretion was increasing already after two hours with U-Calcium/U-creatinine ratio and CCCR respectively 46% and 36% elevated (t-test: p=0.014 and p=0.021, data not shown). This facilitated a concomitant drop in serum concentration of ionized- and total calcium, although not statistically significant after correction of multiple comparisons (p=0.061 and p=0.068, respectively, FIG. 2C). Total calcium was significantly lower after 24 hours (p=0.013) but the concomitant significant reduction in albumin levels (p=0.001) maintained ionized calcium or albumin corrected calcium closer to baseline levels. Interestingly, the decrease in serum calcium prompted an immediate increase in serum PTH (p=0.008) that mobilized calcium by increased bone turnover as indicated by the 24% increase in serum CTX1 (p=0.016, FIG. 2D). Serum calcitonin was elevated during the early hypercalciuric phase but shifted to lower than baseline from 24 hours and onwards and was not normalized at study end (p=0.041).

Moreover, serum 1,25-dihydroxy vitamin D increased considerably with 69% and 89% at 72 and 120 hours after injection (p=0.003, p=0.002) and this late induction of CYP27B1 enzyme activity is timed so it could be secondary to the PTH increase as serum 25-OHD was unchanged (FIG. 2E). There was no significant change in serum- or urinary phosphate or phosphate excretion although an early non-significant tendency towards increased phosphate excretion and subsequent decline in serum phosphate was observed. The half-life of hCG is long and high serum concentrations must still have been available 120 hours after injection, where serum testosterone and estradiol remained high, while serum LH and FSH remained suppressed (FIG. 2). Interestingly, also urinary calcium excretion remained high while total calcium remained low and potentially influenced by decreased serum albumin (p=0.013), suppressed calcitonin and persistently elevated serum PTH ongoing at the end of the study 120 hours after injection.

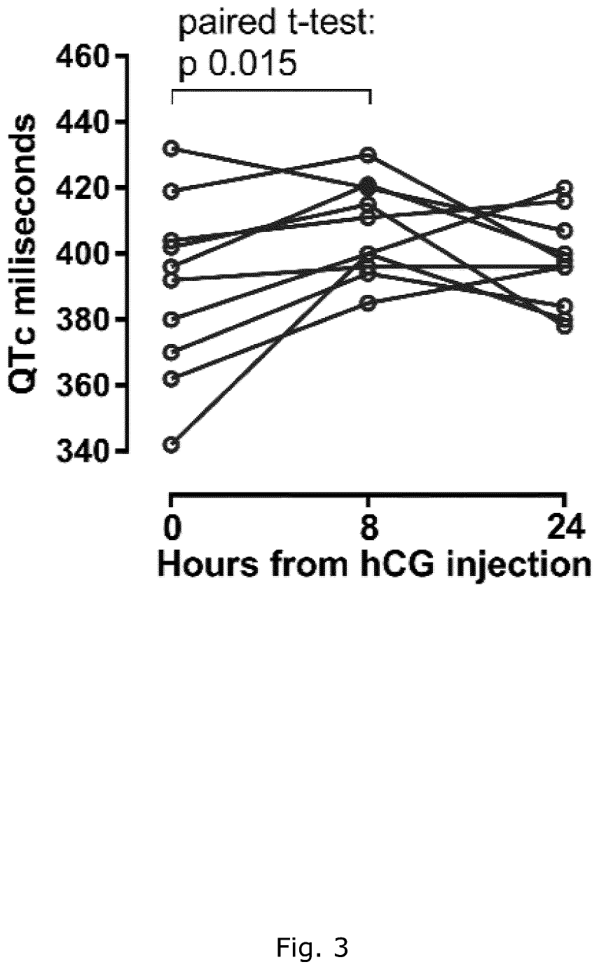

Serum calcium influences heart rhythm and electrocardiograms revealed a significant increase in QTc interval after 8 hours (T0: 340 ms (SD 27 ms) vs. T8: 407 ms (SD 14 ms) p=0.015), but no significant difference in QTc after 24 hours (p=0.362) (FIG. 3).

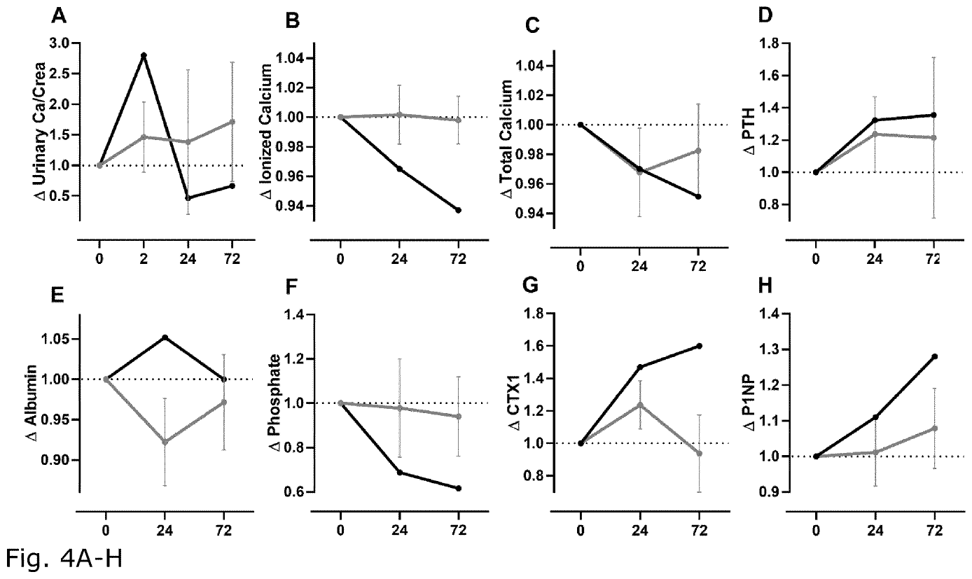

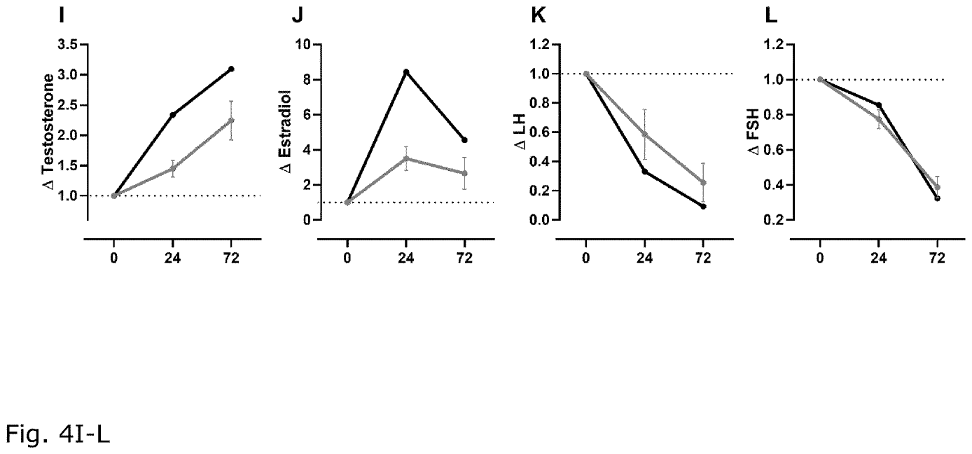

A man with Familial Hypocalciuric Hypercalcemia (FHH), due to an inactivating mutation in CaSR (c.2454G>A, p.Trp818 heterozygote), had comparable baseline serum levels of sex steroids and gonadotropins as healthy men (See Table 1). However, the man with FHH (FIG. 4, black line) experienced a large increase in sex steroids upon hCG injection compared with the 11 healthy men (FIG. 4, grey line) (FIG. 4I-J). FSH and LH decline as a result of the increase in sex steroids and are more than 60% suppressed after 72 hours (FIG. 4K-L). Already 2 hours upon injection his urinary calcium excretion was strikingly increased compared to the increase found in the healthy men (FIG. 4A) and thus resulting in a drastic drop in serum ionized calcium and total calcium after both 24 and 72 hours (FIG. 4B-C).

This facilitated an increase in PTH within the range of the healthy men (FIG. 4D), and a more pronounced decrease in serum phosphate (FIG. 4F). Serum albumin increased after 24 hours (FIG. 4E). The continuing drop in ionized calcium resulted in prolonged and augmented bone turnover, with substantial increase in CTX1, still evident after 72 hours (FIG. 4G), further leading to a potential compensatory bone formation as indicated by increased P1NP (FIG. 4H).

### Conclusion

This study proposes hCG as a novel inducer of urinary calcium excretion and a regulator of calcium homeostasis. It appears that two different LHCGR agonists LH and hCG both can mediate rapid hypercalciuric effects in mice and men, which indicates that it is LHCGR activation rather than the compound that is important. This suggestion is supported by the simple setup in mice with repeated bolus treatments of LH (see example 1) and the single-dose regime with hCG in humans implying that the kinetics of the response and the divergence between LH and hCG is of limited importance for the effect on calcium excretion.

The acute hypercalciuria induced by hCG starts within hours and induces a rapid compensatory increase in serum PTH accompanied by a reduction in serum albumin that effectively rescues circulating ionized calcium within hours and decreases serum phosphate. This comes with an expense as PTH mobilizes calcium from the bone, as illustrated by the secondary increase in CTX, and a very tardy increase in the production of 1,25(OH)2D3 that could indicate that it also could be secondary to other changes for instance sex steroids.

Urinary calcium excretion and PTH remain continuously elevated although no longer significantly different from baseline 120 hours after hCG injection, unlike sex steroids and gonadotropins. The persistent change in calcium homeostasis shows that the renal effects cannot be fully reversed by other classical regulators when high levels of circulating LHCGR agonists are available. Transient changes in minerals are of less clinical importance unless they influence organ function. However, the prolonged QTc interval determined by ECG, the increased bone resorption and persistent hypercalciuria, despite activation of several compensatory mechanisms clearly shows that the changes in calcium homeostasis are potent and persistent enough to induce changes in multiple organs. The organ-specific effects may be direct or indirect but the marked changes in sex steroids seem to be dispensable for the calciotropic effect as urinary calcium excretion increased already on first evaluation whereas serum levels of sex steroids were not altered until after 24 hours. The early observed repression of FSH occurs prior to the rise in sex steroids and may be important although the exact mechanism of action remains speculative and could be due to LHCGR agonists or calcium changes.

CaSR is expressed in the anterior pituitary gland and we speculate that there may be another feedback back loop between LH and calcium homeostasis that also may depend on an intact pituitary-gonadal axis. CaSR seems to be involved in this compensation as a man with inactivating mutations in CaSR had a more dramatic exaggerated effect of hCG on calcium homeostasis that may be due to altered set point in calcium/PTH interaction and could also offer an explanation on chronic versus acute exposure. Calcium is linked with the renin-angiotensin-aldosterone system (RAAS) in vivo and in vitro where acute activation of CaSR inhibits renin release, whereas chronic activation of CaSR is associated with elevated renin-activity thus clearly demonstrating the difference in acute versus chronic exposure.

The hypercalciuric effects of LH and hCG are of biological relevance but the question is whether it is also of clinical relevance. hCG is used by many users of anabolic steroids to stimulate gonadal growth and high LH is a frequent finding in the endocrine clinic in men with hypogonadism or after hemi-orchiectomy. Without being bound by theory when the proposed hypercalciuric mechanism is extended to women, then a therapy blocking LH action will decrease calcium excretion and thereby decrease PTH mediated bone resorption and be a treatment option for osteoporosis. Particularly in perimenopausal and postmenopausal women that have high LH may have beneficial effects of a treatment blocking LH action to reduce renal calcium loss and thereby reduce osteoporosis and the risk of developing primary hyperparathyroidism.

### Example 3—LH and Hypogonadism and Hemi-Orchiectomy

### Aim of Study

The aim of this study was to investigate the relationship between calcium homeostasis and reproductive hormones in healthy, orchiectomized men, and men with testicular/pituitary disturbances. To test a potential interaction, the inventors injected a large dose of hCG and thereby increased endogenous sex steroids and followed the men for subsequent changes in calcium homeostasis.

### Materials and Methods

356 men with various testicular diseases who were all evaluated in our andrological outpatient clinic and referred for hCG stimulation test, were followed longitudinally with blood sampling at baseline and after 72 hours, as per standard protocol. All men were seen for hCG stimulation test between June 2016 and March 2019, and a total of 411 hCG stimulations tests were performed as some men were tested more than once during those years. The 356 men comprised of 110 patients with previous testicular cancer, 28 other types of previous cancer, 57 with gynecomastia, 44 infertile men, 54 men with hypogonadism and the remaining with various testicular/pituitary conditions.

Serum testosterone were analyzed using Access 2 immunoassay system (CV<5%, Beckman-Coulter) and estradiol (CV 13%) was determined by radioimmunoassay (Pantex, Santa Monica, CA). Serum FSH and LH levels were measured with Delfia, CV<5% for both.

Serum and urine total calcium (CV 2.5%) and serum PTH (CV<4%) were all measured using Cobas 8000 while serum ionized calcium (CV 3%) was measured on ABL 837. Longitudinal assessment of blood- and urine samples (delta values) were calculated as fold change, relative to baseline for each person, with all samples' initial value starting at one. All analyses were analyzed with ANOVA with control/baseline as reference value and every p-value presented was adjusted for multiple comparisons with Dunnett's post hoc test.

### Results

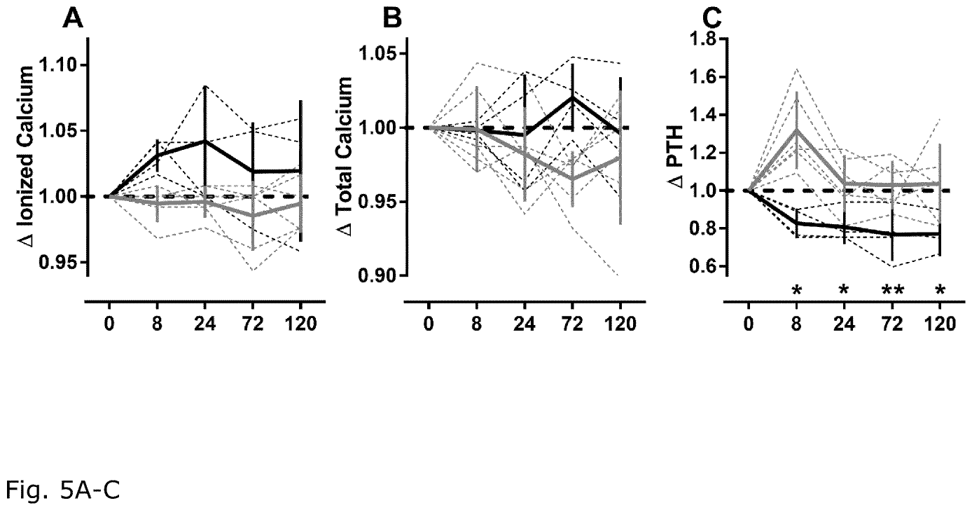

10 hemi-orchiectomized men were injected with 5000 IU hCG. Prior to treatment start, six men had compensatory elevated LH levels due to diminished endogenous testosterone production, whereas four men showed signs of hypogonadotropic hypogonadism/functional hypogonadism with insufficient rise in LH, despite orchiectomy and/or testicular radiation. The proposed calciotropic effect of hCG in 6 men with compensatory elevated LH mimicked that of the healthy men; however, the 4 hypogonadotropic men demonstrated a seemingly paradox reaction upon hCG injection (FIG. 5, Table 2) with a non-significant trend towards increasing serum ionized calcium (FIG. 5A), and total calcium (FIG. 5B) and a significant persistent suppression of PTH throughout the study period (8 hours: p=0.038, 24 hours: p=0.022, 72 hours: p=0.007, 120 hours: p=0.013, FIG. 5C). Interestingly stratification into these two groups would be identical if the men were selected based on baseline PTH rather than LH, since the four hypogonadotropic men also had the highest baseline PTH level (Table 2). This observation is interesting because men with high and low PTH had a comparable increase in testosterone after hCG injection but still very different effects on calcium homeostasis demonstrating independence of sex steroids.

TABLE 2
Baseline level of LH, FSH, testosterone, estradiol and PTH of all
10 men with previously testicular cancer. Men with low LH also
have the highest PTH. The two groups of men have opposite
response in calcium and PTH upon hCG injection.

| | Baseline level | | | | | Δ at 8 hours | |
|---|---|---|---|---|---|---|---|
| | | | Testo | Estra | PTH | Ionized | |
| | | FSH | (nmol/ | (nmol/ | (nmol/ | Cal- | |
| | LH | (IU/L) | L) | L) | L) | cium | PTH |
| Normal | 0.2 | 1.6 | 17.9 | 65 | 6.4 | ↑ | ↓ |
| or low | 0.8 | 2.1 | 1.1 | 127 | 10.6 | ↑ | ↓ |
| LH | 5.0 | 17.2 | 15.0 | 58 | 5.0 | ↑ | ↓ |
| | 7.5 | 7.5 | 9.2 | 105 | 5.7 | ↑ | ↓ |
| Elevated | 10.0 | 31.4 | 12.2 | 90 | 4.3 | ↑ | ↑ |
| LH | 13.6 | 33.4 | 14.1 | 45 | 3.2 | ↓ | ↑ |
| | 16.2 | 37.1 | 16.8 | 84 | 4.2 | ↓ | ↑ |
| | 17.5 | 38.9 | 11.8 | 50 | 3.2 | ↓ | ↑ |
| | 18.7 | 53.0 | 20.5 | 83 | 3.7 | ↓ | ↑ |
| | 20.3 | 50.4 | 21.8 | 79 | 2.9 | ↓ | ↑ |

In a prospective cohort of 356 men with testicular conditions injected with hCG there was no change in serum ionized calcium levels (mean 1.208 mmol/L vs. 1.209 mmol/L, paired t-test p=0.64) nor serum PTH (mean 5.39 pmol/L vs. 5.42 pmol/L, paired t-test p=0.82) determined 72 hours after injection (data not shown).

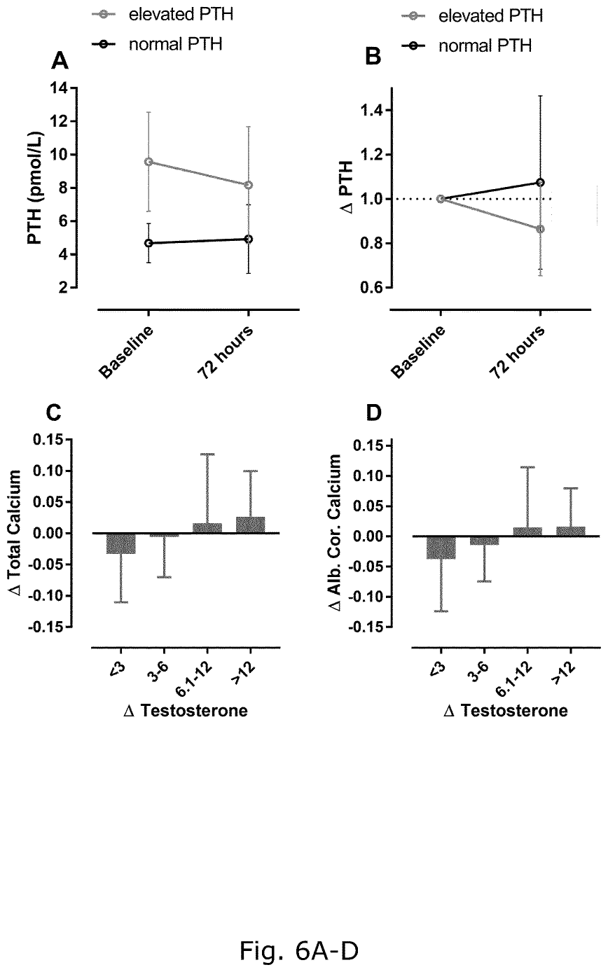

Stratifying the men solely according to their baseline PTH, normal (≤6.9 pmol/L) vs. elevated (>6.9 pmol/L), reproduced exactly the data observed in the clinical intervention of the healthy young men wherein men with normal PTH seemingly had an intermediate drop in serum ionized calcium, leading to a significant, persistent increase in PTH (paired t-test: p=0.039, FIG. 6A-6B). Moreover, baseline PTH above normal range led to a significant, persistent drop in PTH (paired t-test: p=0.002, FIG. 6A-6B). In other words, the impact of hCG on calcium homeostasis is best characterized by the change in serum PTH at 72 hours and showed completely opposite directions in the two groups (high versus normal PTH) (students t-test: p=1.0×10⁻⁵, FIG. 6B), and this was evident in the small clinical intervention and the large prospective cohort.

Despite no persistent change in ionized calcium 72 hours after injection, both total calcium and albumin were significantly associated with the increase in testosterone upon hCG injections. Change in total-(FIG. 6C) and albumin corrected calcium (FIG. 6D) were significantly associated with changes in serum total testosterone after injection (linear regression: Δcalcium/Δtestosterone β 0.002, p=0.019; Δalb. cor. calcium/Δtestosterone β 0.002, p=0.019). Stratifying all the men in four groups based on their increase in testosterone revealed that men with an insufficient increase in testosterone (<3 nmol/L) would have a corresponding drop in calcium. In addition, men with a deficient increase in testosterone (3-6 nmol/L) would have a modest decrease in calcium, whereas men with a sufficient increase in testosterone (6-12 or >12 nmol/L) would experience an increase in serum calcium (total calcium p=0.010 FIG. 6C, albumin corrected calcium p=0.017 FIG. 6D).

### Conclusion

This study shows that high circulating LH prior to injection of hCG is important for the effect of hCG injection on calcium excretion. High LH prior to injection induces a similar response to men with normal LH but men with low LH have an aberrant and atypical response. This indicates that patients with high LH respond in a similar way with increasing calcium excretion and high serum PTH leading to high bone resorption. The organ-specific effects may be direct or indirect but the lower the testosterone change the larger effect on calcium homeostasis was observed. This indicates that high LH with no increase in sex steroids as you experience during menopause or gonadal failure aggravates the loss of calcium in the urine and thereby increases the risk for osteoporosis. Men or women with gonadal impairment cannot compensate with increasing sex steroids required to rescue the calciuric effect of LH surge, which may be critical in men with primary hypogonadism and postmenopausal osteoporosis.

To conclude, high LH is a frequent finding in the endocrine clinic in men with hypogonadism or after hemi-orchiectomy. Further, when the proposed hypercalciuric mechanism is extended to women, then a therapy blocking LH action may decrease calcium excretion and thereby decrease PTH mediated bone resorption and be a treatment option for postmenopausal osteoporosis and symptoms related to perimenopause and postmenopause induced by the increased serum LH levels.

### Example 4—Ex Vivo Culture of Human Kidney Tissue

### Aim of Study

The aim of this study was to investigate the gene expression of LHCGR exon 11 (encodes the intracellular part of LHCGR) and LHCGR exon 2-4 (encodes the extracellular part of LHCGR) in response to exposing the kidney samples to treatments with vehicle, PTH, LH, hCG, estradiol and testosterone for three hours. In addition, the aim of said study was also to investigate the gene expression of TRPV5 (Transient receptor potential cation channel subfamily V member 5-a calcium channel protein) in response to treating the kidney samples with vehicle, PTH, LH, hCG, estradiol and testosterone for three hours or 24 hours.

### Materials and Methods

### Human Kidney Tissue Cultures:

Human kidney tissues from 5 adults (4 men, 1 woman) were cultured. All donors underwent partial nephrectomy due to suspected malignant kidney tumors and a pathologist removed tissues outside the malignant area. Kidney specimens were dissected in small pieces 1 mm³ and cultured for either 3 or 24 hours in a hanging drop ex vivo model. Tissues were treated with either vehicle (media containing DMEM, insulin-transferrin-selenium, penicillin, streptomycin and 10% fetal bovine serum) or media also containing one of the following: PTH (0.01 pg/mL), LH (1 IU/mL), hCG (1 IU/mL), estradiol (1 nmol/L) or testosterone (50 nmol/L) and subsequently stored at −80° C. for subsequent RNA purification and cDNA synthesis.

Gene expression profiling was done by qRT-PCR analyzing levels of calcium transporters SLC12A-1, SLC12A-2, TRPV5 as well as calcium-sensing receptor (CASR). The inventors also measured kidney expression of exon 2-4 and 11 of LHCGR using a primer-sequence previously validated by sequencing of the product from both serum and testis tissue. As calcium transporters are differentially expressed in the different parts of the nephron, all samples were standardized according to the expression of Uromodulin (UMOD), a protein located in the thick ascending limb of the loop of Henle and subsequently to MAP3K7 expressed in the distal convoluted tubules and in addition to being standardized to the housekeeping gene, RPS20. Ethical committee approval: H-17004362. All samples were analyzed with ANOVA with control/baseline as reference value and every p-value presented was adjusted for multiple comparisons with Dunnett's post hoc test.

### Results

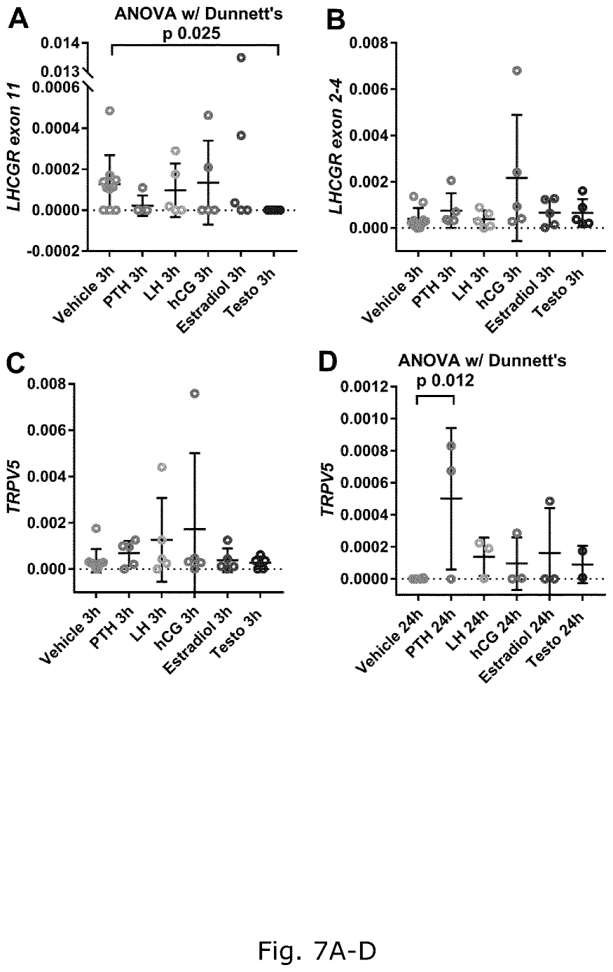

LHCGR was expressed in most human kidney specimens cultured with most CT values below 34 despite variable RNA content in the samples using two different primers. Evaluation of three hours ex vivo cultures showed that the transmembrane part of LHCGR mediating intracellular signaling (exon 11) was suppressed by testosterone treatment in all kidney samples (5 out of 5 below detection) compared to vehicle (3 out of 10 below detection) p=0.025 (FIG. 7A). While there was no significant change in the expression of the extracellular part (exon 2-4); however, a tendency towards increased expression of these exons was found after hCG treatment (p 0.059, FIG. 7B). Expression of TRPV5 after 3 hours of culture was unaffected (FIG. 7C). However, after 24 hours TRPV5 expression was significantly increased by PTH (p 0.012) (FIG. 7D). Neither expression levels of the CASR or calcium transporters SLC12A-1, SLC12A-2 nor SLC12A-3 were influenced by any of the treatments and adjusting levels of expression for UMOD or MAP3K7 in each sample did not alter results compared with adjustment for standard housekeeping gene.

### Conclusion

LH or hCG treatment induced no transcriptional changes of some of the main calcium transporters. LHCGR was abundantly expressed in the kidney of both sexes and testosterone downregulated LHCGR expression level in the kidney of both sexes.

The conclusion of the study is that LHCGR is highly expressed in the kidney which indicates that LH and hCG can induce direct renal effects. The suggested influence on calcium excretion seems to not be mediated by transcriptional regulation of the main calcium transporter (encoded by TRPV5) but may be through translation or protein abundance in the membrane.

### Example 5—Reduce Visceral Adiposity and Improve Energy Homeostasis

### Aim of Study

High LH levels are possibly responsible for fat accumulation and redistribution in postmenopausal women, pregnant women, and men with increased LH levels. Therefore, the aim of the present study was to investigate whether it was possible to reduce visceral adiposity and improve energy homeostasis by targeting LH.

### Materials and Methods

The human-derived adipocyte-cell-models used were: hMADS (from a subcutaneous depot), human Simpson-Golabi-Behmel syndrome (SGBS) preadipocyte cell strain, and TERT-hWA (from a subcutaneous neck depot). They were propagated and differentiated as briefly described here: All cells were cultured until two days post-confluence (designated day 0) where they were induced to differentiate in advanced DMEM/F12 supplemented with 2% fetal bovine serum (FBS) (Life Technologies), L-glutamine (2 mM) (Life Technologies), penicillin (62.5 μg/mL), streptomycin (100 μg/mL) Sigma-Aldrich), insulin (5 ug/mL) (Roche), examethasone (1 μM) (Sigma-Aldrich), 3-isobutyl-1-methylxanthine (0.5 mM) (Sigma-Aldrich), rosiglitazone (1 μM) (Cayman Chemical), human cortisol (1 μM) (Sigma-Aldrich) and T3 (1 nM) (Sigma-Aldrich). At day 3, the medium was refreshed with the same medium used at day 0. At day 6 and 9, the cells received Advanced DMEM/F12 supplemented with 2% FBS. At day 12, the adipocytes were defined as mature, and the percentage of differentiated cells was 50-70% based on microscopic inspection irrespective of cell model. RNA was extracted for cDNA synthesis from undifferentiated (designated day 0) and differentiated (day 12) cells and PCR was conducted using specific primers. All analyses were analyzed with student t-test or ANOVA using the corresponding control/baseline as reference value. Every p-value presented was adjusted for multiple comparisons with Dunnett's post hoc test.

### Results

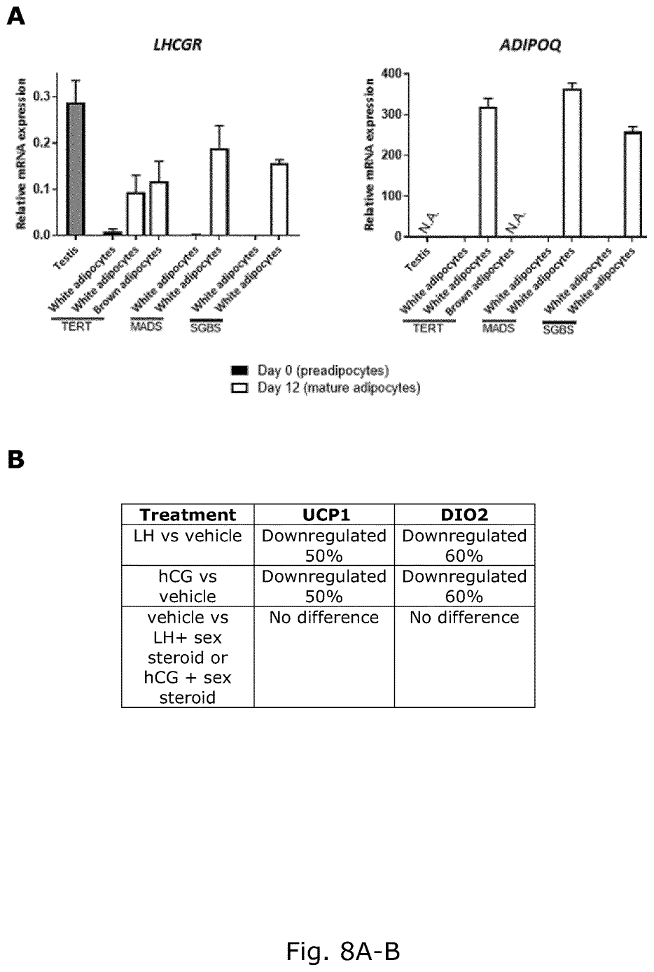

LHCGR was expressed in all mature fat cells and in the brown fat cells (FIG. 8A). This indicates a plausible direct effect of LH and hCG. Expression of ADIPOG proved that the fat cells differentiated. Two doses of LH and two doses of hCG were tested and showed that UCP1 and DIO2 were downregulated in TERT cells compared with vehicle-treated cells after 48 hours exposure (p<0.05) (FIG. 8B). Expression level of LHCGR in mature fats cells were between 30-50% of the expression level found in the testicle (FIG. 8A).

### Conclusion

This study shows that LHCGR is expressed in both mature brown and white adipocytes at a relatively high expression level compared with the testis wherein the LHCGR is abundantly expressed. The presence of the receptor suggest a direct effect of LH and hCG, which was proven in TERT cells where both LH and hCG treatment suppressed UCP1 and DIO2 expression. UCP1 is an uncoupling protein that is critical for heat generation in fat cells and low activity implies less heat generation and a drop in core temperature, which is what postmenopausal women experience. Low UCP1 levels will lead to increased storage of fat instead of heat generation and interestingly this effect is only present in low sex steroid levels which is a hallmark of osteopororis. These results indicate that high LH may facilitate less energy expenditure and thus increased obesity. Moreover, this phenotype may be amplified by the LH and hCG mediated suppression of DIO2, which is the essential enzyme responsible for the vast majority of the conversion of thyroid hormones from the inactive T4 into the active T3. T3 in circulation is not always a good marker for thyroid action—the local concentrations in the target tissue is a better measure. Low activity of DIO2 will lead to low conversion into T3 and may induce hypothyroidism that will worsen the hot flashes and impair thermogenesis as thyroid hormones also influence brown fat cell activity and central heat regulation and thereby also hot flashes and night sweats. This will also increase obesity as less energy will be consumed due to less brown fat usage. Thus, the conclusion of this study is that LHCGR is highly expressed in mature adipocytes and high LH may increase obesity directly by suppressing heat generation through UCP1 and thyroid hormone conversion into T3 that will increase obesity. Hence, it is believed that LH and hCG reduce brown fat mass and thermogenesis and thyroid hormone conversion resulting in increased white cell adiposity. In sum, the data presented here indicates that white cell adiposity may be improved by treatment with an LH antibody.

### Example 6—LH and hCG Reduce Brown Fat Mass, Conversion of Thyroid Hormones T4 to T3 and Thermogenesis

### Aim of Study

The aim of the present study was to investigate whether LH reduces brown cell adipocyte function and mass and thereby lead to less energy spenditure on heat and more on storage.

### Materials and Methods

Eight weeks old male wild type mice (C57BL/6) caged at Biotest Facility, Trige, Denmark, were treated for 10 days with either vehicle (n=12; NaCl 0.9%×1 daily s.c.), LH (n=8; 66.7 IU/kg×1 daily s.c.), or hCG (n=8; 666.7 IU/kg, every other day, i.p.). At termination, blood and fat sampling was performed, animals were sacrificed and body weight was assessed. Danish Animal Experiments Inspectorate License no. 2019-15-0201-00236. All analyses of mice serum, urine, and organ weight were analyzed with ANOVA with control/baseline as reference value and every p-value presented was adjusted for multiple comparisons with Dunnett's post hoc test.

The human-derived adipocyte-cell-models used were: TERT-hWA (from a subcutaneous neck depot) and TERT-B (from a brown fat cell depot). They were propagated and differentiated at day 12 where the adipocytes were defined as mature, and the percentage of differentiated cells was 50-70% based on microscopic inspection irrespective of cell model. They were subsequently treated with different dosages of LH and hCG and compared with the vehicle samples. RNA was extracted for cDNA synthesis and PCR was conducted using specific primers. All analyses were analyzed with t-test or ANOVA using the corresponding control/baseline as reference value and every p-value presented was adjusted for multiple comparisons with Dunnett's post hoc test.

### Results

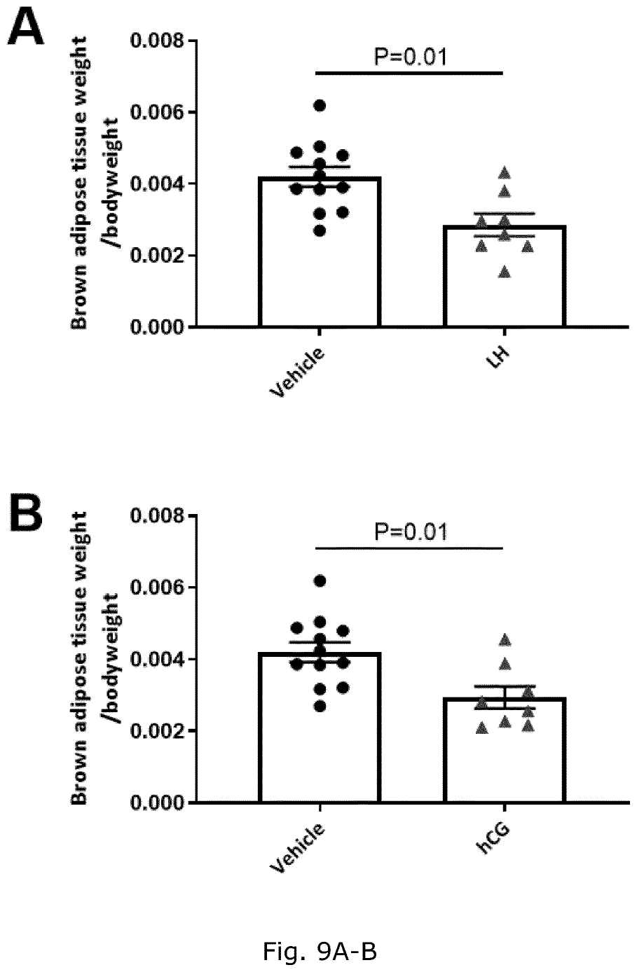

High and repeated dosing of LH (FIG. 9A) or hCG (FIG. 9B) of adult mice reduced brown fat mass significantly. This effect may be direct or indirect as LH and hCG also induce sex steroid production. A direct effect is plausible as our data show that LHCGR is expressed in mature human brown and white adipocytes but virtually not in immature white adipocytes.

### Conclusion

In addition to the in vitro effect shown in example 5, LH and hCG also have a potent in vivo effect which was tested by injection of LH or hCG for 2 weeks in wildtype mice. The injection of LH and hCG decreased brown adipocyte cell mass compared with the vehicle treatment. This observation is in line with the in vitro data presented in example 5 showing that the suppressive effect on thermoregulation and thyroid hormone conversion may lead to less brown cell activation and less energy consumption. This will in a more chronic phase lead to less brown adipocyte cells which is what we see here following two weeks treatment with either LH or HCG.

The conclusion of the study is that high LH and hCG may increase obesity directly by suppressing heat generation, thyroid hormone conversion and brown adipocyte activation leading to less brown adipocytes because the activity drops. Instead of heat generating cells, white adipocytes that can store energy will be generated.

### Example 7—Injection of Chorionic Gonadotropin (hCG) in Healthy Men to Study Effects on Adrenal/Pituitary Function, Thyroid Hormones and Metabolism

### Aim of Study

LHCGR is present in the adrenal gland in adipocytes. Thus, LH or hCG may affect the production of adrenal steroids such as cortisol, aldosterone, androgens and metabolic factors such as insulin and glucose homeostasis. The aim of this study was therefore to test a potential interaction by injecting a large dose of hCG and thereby increase endogenous sex steroids and follow the test subjects for subsequent changes in adrenal hormones and metabolic factors.

### Materials and Methods

### Cohorts:

### Clinical Intervention

11 healthy men and 10 men with previous testicular cancer and hemi-orchiectomy and/or testicular radiation were invited to participate in a prospective clinical trial, receiving 5000 IU hCG (Pregnyl) intramuscularly. They were followed longitudinally with blood- and urine sampling at baseline and 2, 8, 24, 72 and 120 hours after injection. All samples were drawn while fasting, except after 8 hours. Sampling and subsequent injection at baseline and sampling at 24, 72, and 120 hours were done between 8.00 am and 9.30 am. All analyses were analyzed with ANOVA with control/baseline as reference value and every p-value presented was adjusted for multiple comparisons with Dunnett's post hoc test. However, evaluation of changes in QTc interval on ECGs was available only at two time-points and therefore statistically analyzed by paired T-test.

### Results

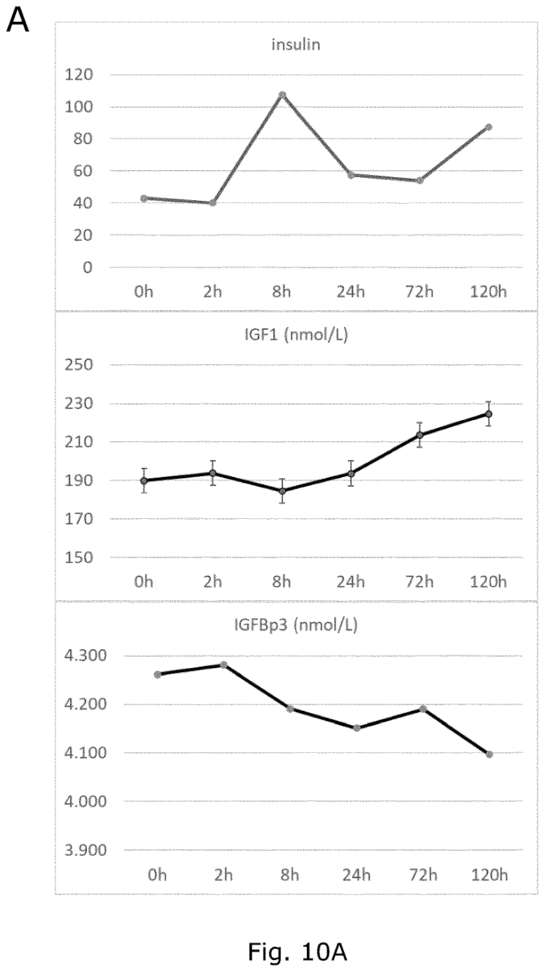

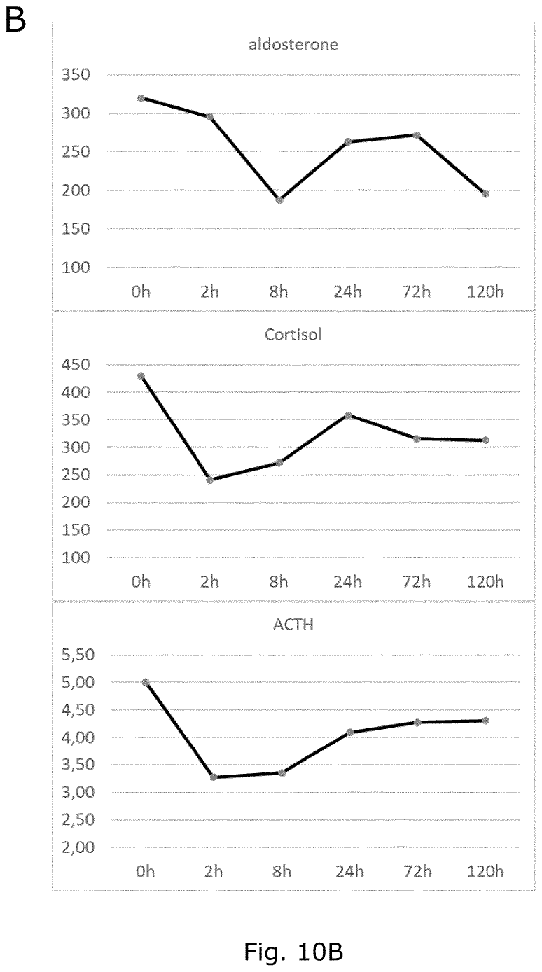

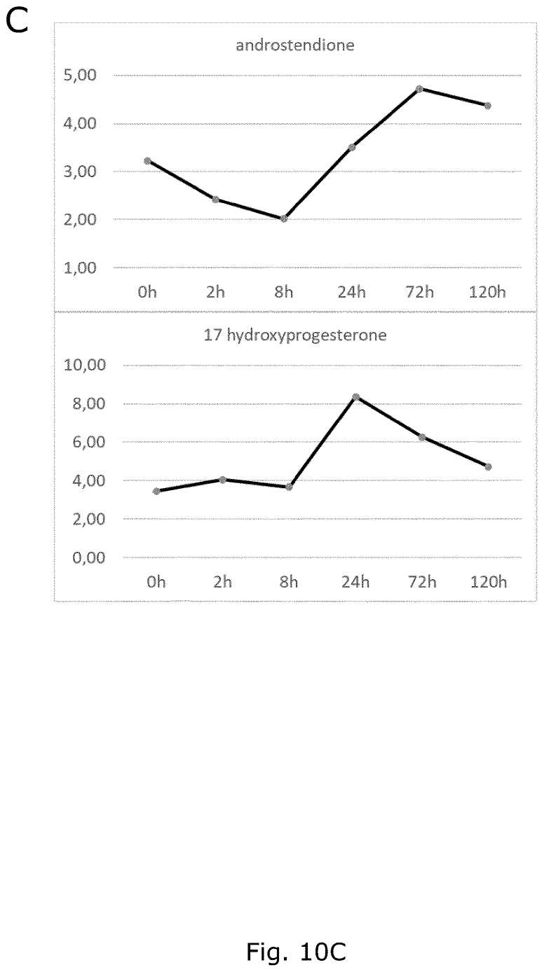

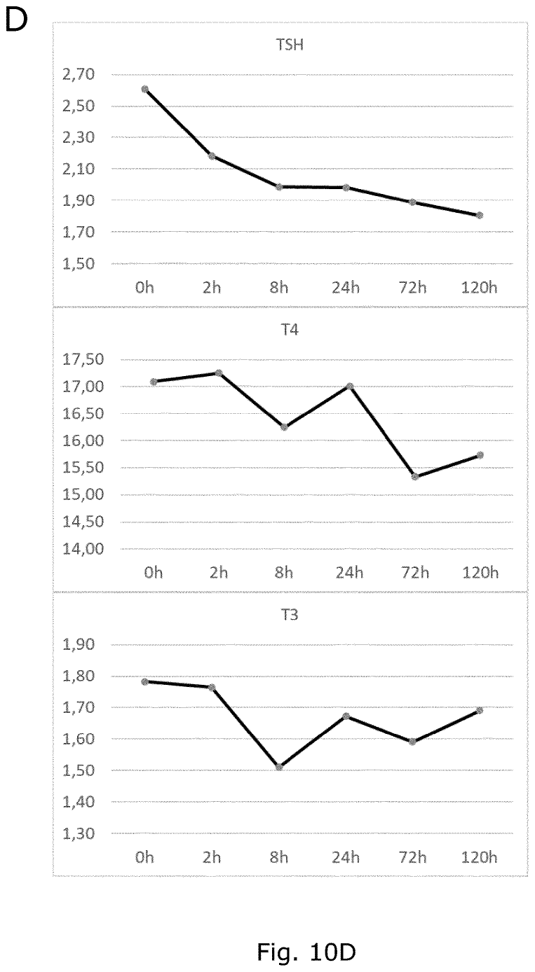

hCG injection induced a persistent (after 120 hours) increase in fasting insulin to up to 100% and in serum IGF1 of 18% and suppressed the binding protein IGFBP3 with 3% resulting in higher free IGF1 levels (FIG. 10A). These changes will influence metabolic function and may ultimately lead to increased insulin insensitivity and type 2 diabetes. hCG injection also induced a marked change in steroid hormone production that at least partly was due to a pituitary effect and to some degree an adrenal effect. hCG induced a suppression of aldosterone and cortisol of 38 and 27%, respectively, that may be due to a pituitary effect as ACTH was suppressed with 17% (FIG. 10B). However, hCG induced adrenal production of androstendione and 17 hydroxyprogesterone with >50% and 100%, respectively, which shows an effect of hCG on adrenal function which clearly indicates a stimulatory effect that differs from the suppressive effect on the pituitary gland (FIG. 10C). The effect of hCG on thyroid hormones may be on DIO2 expression in the fat that may explain the impact on T3 and T4 that is 5-8% lower after hCG stimulation. However, the most pronounced effect is on TSH that drops 30% which in a low T3 and T4 situation clearly shows a suppressive effect on TSH production (FIG. 10D).

### Conclusion

This study suggests a basal physiological, yet undescribed endocrine crosstalk between hCG/LH and the pituitary, adrenal gland and metabolism proposing LH and hCG as novel suppressors of pituitary function and inducers of andronstendione production which may lead to increased hair problems in postmenopausal women. The suppressive effect may be important and lead to less cortisol production that may induce fatigue which is a common problem in postmenopausal women. Moreover the suppressive effect on TSH may lead to hypothyroidism and impaired thermogenesis. Without being bound by theory, when the proposed mechanism is extended to women, then a therapy blocking LH action will alleviate suppressive function of pituitary function (ACTH, cortisol, and TSH) reduce hair growth in genital, stomach and face of postmenopausal women and improve insulin sensitivity and not least hypothyroidism in patients not adequately treated with eltroxin (T4). Particularly perimenopausal and postmenopausal women that have high LH, may have beneficial effects of a treatment blocking LH action.

### Example 8—Investigating the Effect of Inhibiting LH-β, LHCGR or GnRH in a Menopausal Mouse Model

### Aim of Study

The aim of the study was to investigate the effect of an LH-β antibody (ab), LHCGR ab or a GnRH antagonist in ovariectomized female mice by measuring temperature and tissue weights of said ovariectomized female mice following treatment with IgG2b (control) or 1 of 3 test articles (LH-β ab, LHCGR ab or GnRH antagonist).

### Materials and Methods

### Generation of Specific Antibodies Targeting Mouse LH Beta or LHCGR

Antibodies were produced by ABClonal.

Monoclonal mouse antibodies (IgG2B; Kappa) were generated by raising antibodies against specific epitopes in:
LH beta amino acid sequence; listed as SEQ ID NO: 1
LHCGR amino acid sequence; listed as SEQ ID NO: 2

Testing and validation of the antibodies were also conducted by the inventors.

### LH Antibody:

As the sequence identity is high with thyrotropin subunit beta precursor, immunization was done using a recombinant protein fragment corresponding to amino acids S21-L141 of SEQ ID NO: 1 for immunization.

This antigen meets the requirements of antibody preparation, with suitable epitope linearity, hydrophilicity, immunogenicity, and epitope exposure and was expressed in an E. coli system.

The produced and tested LH targeting antibody has the following sequences:

### Variable Light Chain

CDR1 according to SEQ ID NO: 3;
CDR2 with the amino acid sequence KVS; and
CDR3 according to SEQ ID NO: 4;
An overall variable Light chain sequence according to SEQ ID NO: 5 (comprising the CDR sequences).

### Heavy Chain:

CDR1 according to SEQ ID NO: 6;
CDR2 according to SEQ ID NO: 7; and
CDR3 according to SEQ ID NO: 8;
Overall Heavy chain sequence according to SEQ ID NO: 9 (comprising the CDR sequences).

### LHCGR Antibody:

As the sequence identity is high with leucine-rich repeat-containing G-protein coupled receptor 5 precursor, immunization was done using a recombinant protein fragment corresponding to the extracellular part comprising amino acids P27-G362 of SEQ ID NO: 2 for immunization.

This antigen meets the requirements of antibody preparation, with suitable epitope linearity, hydrophilicity, immunogenicity, and epitope exposure and was expressed in an E. coli system.

Following antigen preparation using E. coli, immunization was conducted in 5 mice (Balb/C) that also received an immune stimulatory cocktail because the antigens were mice antigens. The immune responses were tested by Enzyme-linked immunosorbent assay (ELISA) and serum from three mice with good immune response were used for immunohistochemistry (IHC) on mouse pituitary or testis tissue to determine specificity with alpha GSU and CYP17B1, respectively. The best and most specific mice were selected, and fusion of candidates' immune cells was conducted. Subsequently, screening for positive hybridomas by ELISA and positive supernatants were again validated in target organs by IHC and Western blot. Quality of subcloning, expansion, and confirmation of positives were ensured by ELISA and afterwards antibodies were purified from hybridoma supernatant and tested using IHC and Western Blot before in vivo usage.

### GnRH Antagonist:

The GnRH antagonist Cetrorelix (Merck) was used in the present example.

### Study Groups

Bilateral ovariectomized female C57BL/6JRj mice, 8 weeks of age, were included in the study and administered with IgG2b (control) or 1 of 3 test articles (LH-β ab, LHCGR ab or GnRH antagonist) either at study day 1, or once a day for 9 days. The control/vehicle treatment was a non-specific antibody (IgG2b). The route of administration was through intraperitoneal (i.p.) injection. The study had four groups in total (Table 3); group 1 (female, n=6) was treated i.p. with IgG2b (control) on study day 1 and day 5, group 2 (female, n=3) was treated i.p. with LH-β ab at study day 1, group 3 (female, n=3) was treated i.p. with LHCGR ab at study day 1, group 4 (female, n=3) was treated i.p. with GnRH antagonist once a day (q.d.) for 9 days. Blood for preparation of serum was collected prior to dosing on study day 1 and at termination on study day 10. Core temperature and skin temperature were determined in female mice pre-dose on study day 1, at study day 5 and at termination on study day 10.

TABLE 3
Schematic illustration of the four (female mice) treatment groups

| Group | Mice | | Conc. | Dose | Dose volume |
|---|---|---|---|---|---|
| No. | (n) | Treatment | (mg/mL) | (mg) | (mL) |
| 1 | 6 | Control (IgG2b) | 0.34 | 0.17 | 0.5 |
| 2 | 3 | LH-β ab | 0.34 | 0.17 | 0.5 |
| 3 | 3 | LHCGR ab | 0.34 | 0.17 | 0.5 |
| 4 | 3 | GnRH antagonist | 0.02 | 0.5 ug/g | 25 mL/kg |

### Ovariectomized Female C57BL/6JRj Mice

Seventeen (17) ovariectomized C57BL/6JRj female mice 8 weeks of age were supplied by Janvier Labs France. The ovaries from the female mice were surgically removed by the supplier. The mice were caged in European IVC cages type IIL. Temperature was 20° C. to 24° C. and the light cycle 12-hour dark and 12-hour light (lights on 06.00). The diet was Altromin 1324, produced by Altromin, Im Seelenkamp 20, 32791 Lage, Germany.

### Measurements

Temperature measurements were done using rectal thermometer (thermometer BIO-TK8851 combined with rectal probe BIO-BRET-3, both from BIOSEB Lab instruments) for core temperature and infrared (153-IRB Infrared Thermometer, BIOSEB Lab instruments) for skin temperature. The core temperature was recorded rectally, and skin temperature was recorded at the tail and the head between the ears at the same time of the day (morning). Immediately after termination, tissues (fat and thyroid mass) were dissected, weighed, placed in Eppendorf tubes and snap frozen by emerging the tube in liquid nitrogen or placed in histology jar and fixed in 4% neutral buffered formalin.

### Results

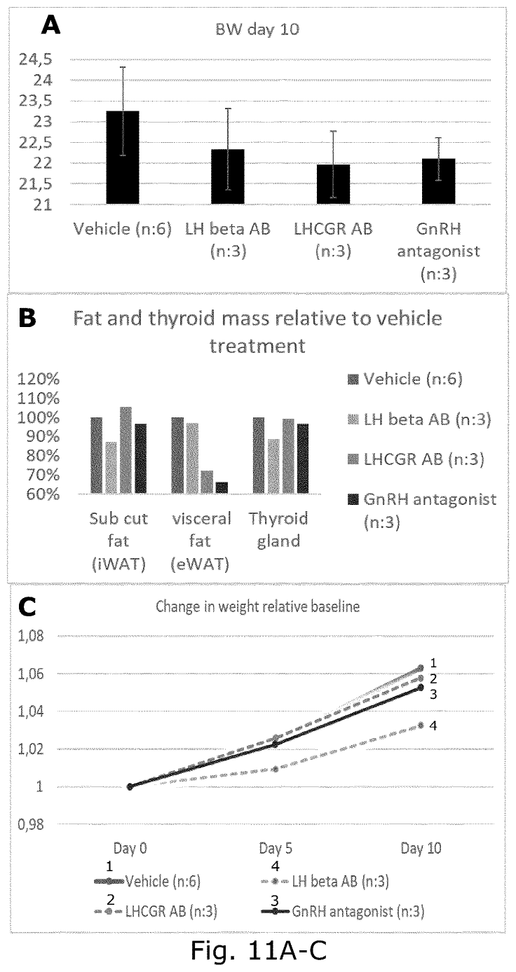

Injection of antibodies targeting LH-β or LHCGR changed body weight, fat mass and thyroid mass more than the GnRH antagonist or vehicle treatment in the present mouse model of female menopause (FIG. 11).

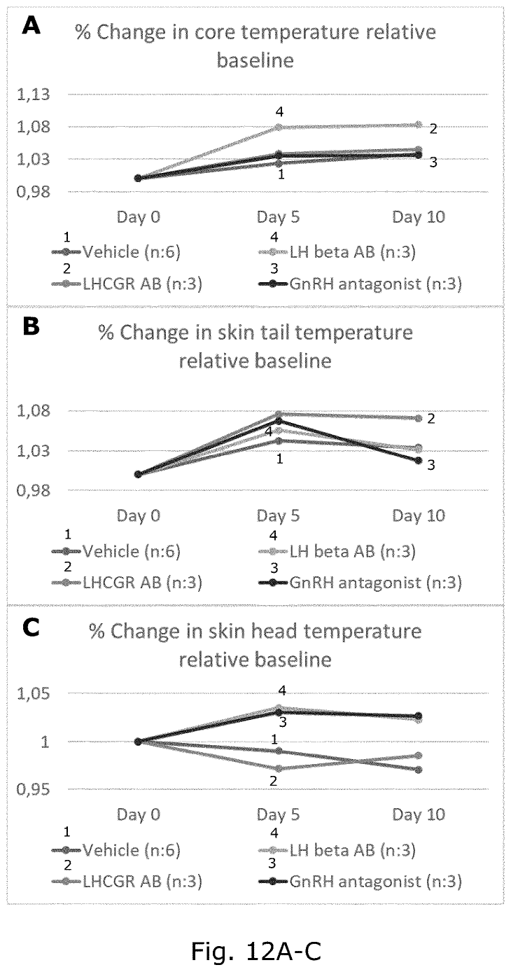

Body weight was lower in mice treated with antibodies targeting LH-β or LHCGR compared with both vehicle treatments. Body weight was also lower in mice treated with GnrH antagonist suggesting that all treatments lowering LH activity had lower body weight after 10 days treatment compared with vehicle treatment (FIG. 11). Interestingly, LH and LHCGR antibodies and GnRH antagonist suppressed particularly visceral fat mass, but the LH antibody also suppressed subcutaneous fat mass, which explains the lower body weight of said treatment group although there was also a small difference in body weight from the beginning of the study. Therefore, the change in body weight following treatments with antibodies targeting LH or LHCGR and GnRH antagonist versus vehicle were determined and showed that especially antibodies against LH were effective in suppressing the effect of menopause (gonadectomy) on weight induction. The effect was most pronounced in the first 5 days. Moreover, antibodies against LH also lowered thyroid mass, which may suggest that by inhibiting the LH suppressive effect on thyroid conversion from T4 to T3 and this may lead to less TSH and lower thyroid gland mass. Interestingly, the LH-β ab increased the core temperature by 8% relative to baseline, whereas the other treatments (including the vehicle) only increased the cores temperature by approximately 3% on day 5 and 10 (FIG. 12). The treatments also affected the skin temperatures, however, said changes were not as pronounced.

### Conclusion

This study suggests that blocking LH action either by blocking LH-β or the receptor LHCGR with antibodies reduces the weight gain you normally experience following menopause. GnrH antagonist that also blocks LH action has also a reducing effect on postmenopausal weight gain and fat storage that support a role role of LH. This is demonstrated by the lower bodyweight but also by showing that the relative increase is suppressed particularly in antibodies targeting LH-β. The lower body weight can be explained by reduced visceral fat mass for all treatments and also subcutaneous fat for LH-β antibody. This clearly indicates that blocking LH influence adipocyte function, and adipocytes are also important for thyroid hormone conversion and temperature regulation. The Antibody against LH-β also suppressed thyroid volume, which supports that blocking LH increases T3 and thereby lowers TSH and the resultant lower thyroid mass. The increase in T3 could in theory also explain the 8% increase in core temperature following blocking LH activity. However, it is unknown whether the effect of LH blocking therapy on core temperature is mediated by influencing heat generation by fat cells or by central regulation. The 8% higher increase in core temperature may be important as core temperature drops after menopause and has been linked with hot flashes. The influence on core temperature is rapidly mediated and is also seen although with less potency using the LHCGR antibody. All LH blocking treatments also increased skin temperature more than the vehicle treatment did. Noteworthy, the increase in temperature cannot be due to an immunological reaction since the vehicle group also received an antibody that would induce a similar immunological response.

Thus, to summarize, this study demonstrates that blocking (with either antibodies targeting LH or LHCGR or using a GnrH antagonist) circulating high levels of LH in menopause diminishes weight gain, visceral and subcutaneous fat accumulation, lowers thyroid goiter (irregular growth of the thyroid) by improving thyroid function and increases core and skin temperature that will reduce the incidence and severity of hot flashes.

### Example 9—LH Increases Visceral Adiposity and Impairs Brown Fat Cell Function

### Aim of Study

High LH levels are possibly responsible for fat accumulation, less heat generation from brown fat cells and redistribution of fat in postmenopausal women, and pregnant women. Therefore, the aim of the present study was to investigate the main genes and cellular pathways influenced by LH in white and brown fat cells.

### Materials and Methods

The human-derived adipocyte-cell-models used were: hBa (from a subcutaneous depot), human Simpson-Golabi-Behmel syndrome (SGBS) preadipocyte cell strain, and TERT-hWA (from a subcutaneous neck depot). They were propagated and differentiated as described in Example 5.

Brown adipocytes were exposed to different dosages of LH and hCG with and without sex steroids in the media and compared with the vehicle samples. RNA was extracted for cDNA synthesis and RNA seq (core facility and Bioinformatics assistance from CPH University) was performed supported by PCR using specific primers. All analyses were analyzed with t-test or ANOVA using the corresponding control/baseline as reference value and every p-value presented was adjusted for multiple comparisons with Dunnett's post hoc test.

### Results

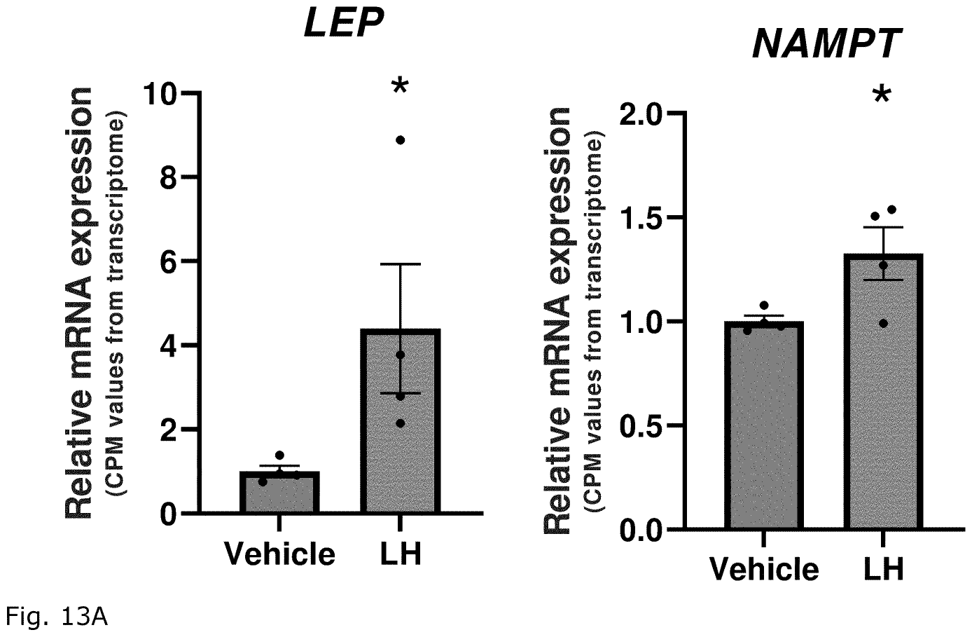

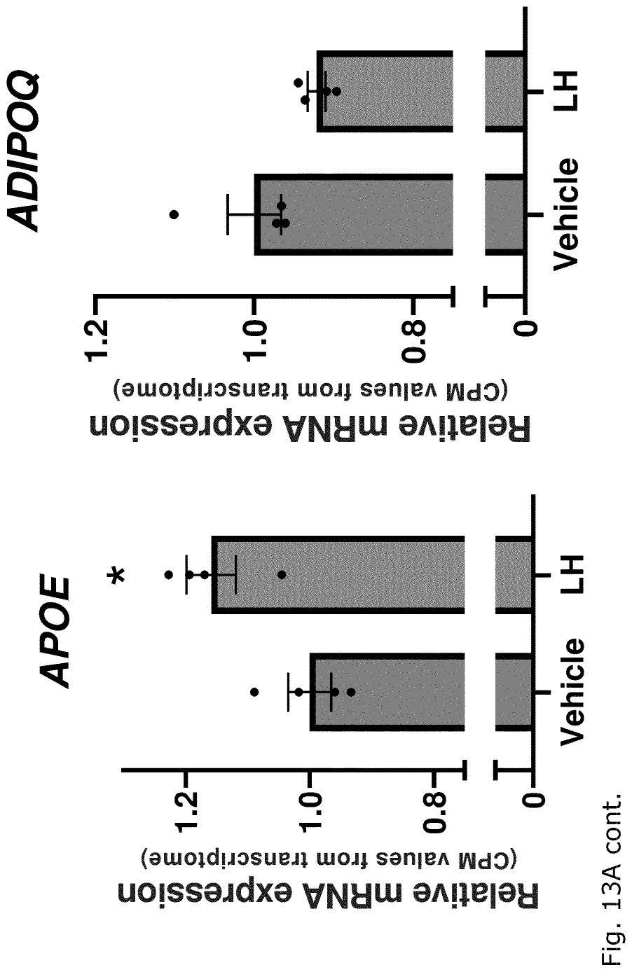

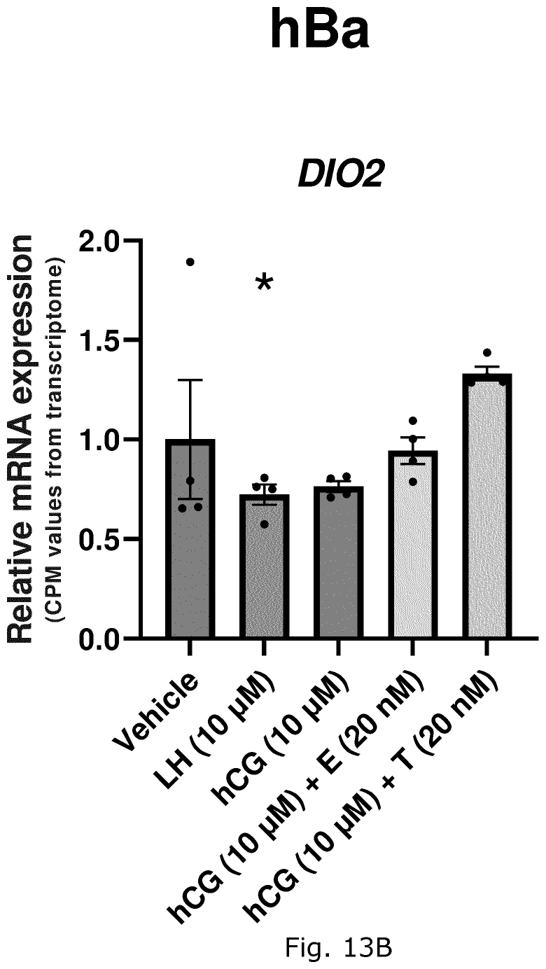

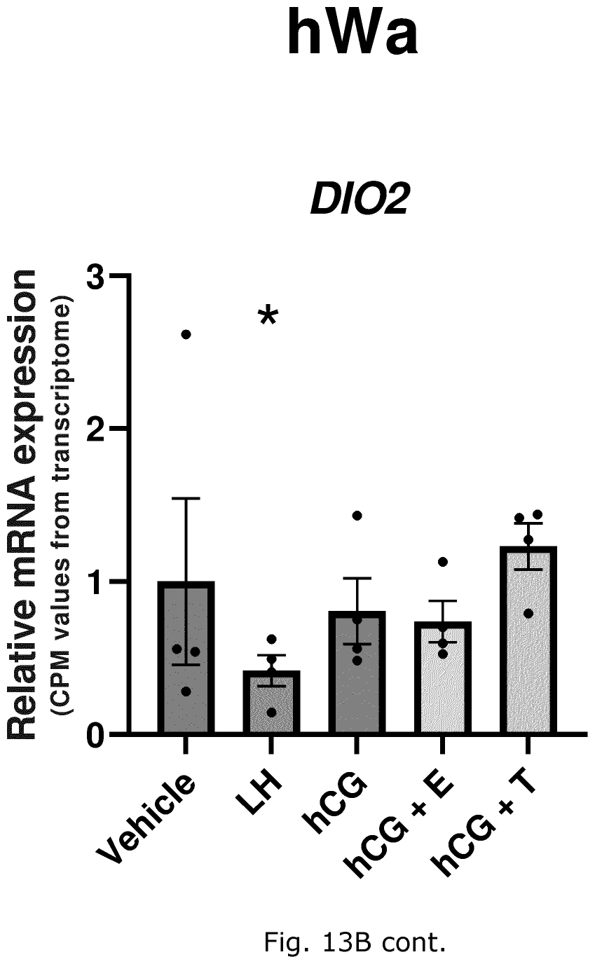

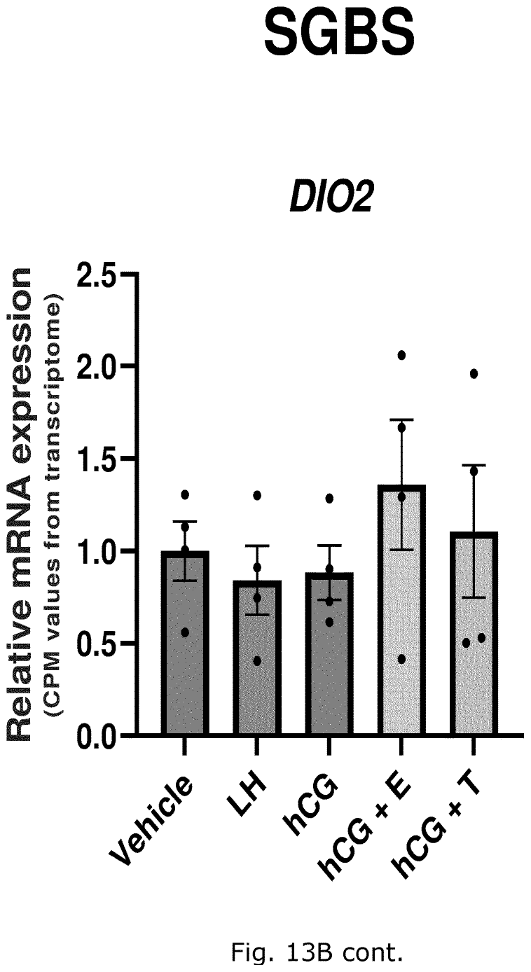

LHCGR was expressed in all mature fat cells and in the brown fat cells. This indicates a plausible direct effect of LH and hCG. In the white fat cells, LH influences mitochondrial function by suppression of multiple signaling systems important for mitochondrial function and both aerobe and anaerobe metabolism including the cytochrome system, electron transport, NADH synthesis, Krebs cycle, fatty acid metabolism, thyroid hormone metabolism, prostaglandins etc. Interestingly, cold-induced thermogenesis was suppressed in both white and brown fat cells when exposed to high LH. In addition to the effect on major signaling pathways, more than 400 genes were directly influenced by LH. Some of them are highlighted in Table 4 and 5 and FIG. 13 and include Leptin, Nampt, APOE and adiponectin that all are essential for adipocyte function and obesity.

TABLE 4
Changes in signaling pathways using RNA seq data from
white adipocytes exposed to vehicle or LH treatment.

| | | Number of | |
|---|---|---|---|
| Signaling pathway | Direction | genes | P value |
| Fatty acid beta-oxidation | Down | 73 | 1.80E−06 |
| Lipid oxidation | Down | 97 | 3.00E−04 |
| Fatty acid oxidation | Down | 95 | 5.10E−04 |
| Fatty acid catabolic process | Down | 97 | 2.40E−05 |
| Positive regulation of fatty acid | Up | 18 | 2.00E−05 |
| transport | | | |
| Thyroid hormone metabolic | Up | 16 | 4.30E−04 |
| process | | | |
| Positive regulation of cold- | Down | 78 | 4.40E−04 |
| induced thermogenesis | | | |
| Aerobic respiration | Down | 172 | 6.93E−09 |
| Aerobic electron transport chain | Down | 172 | 1.94E−05 |
| Oxidative phosphorylation | Down | 127 | 2.13E−08 |
| Mitochondrial ATP synthesis | Down | 85 | 6.95E−08 |
| coupled electron transport | | | |
| Mitochondrial electron transport, | Down | 49 | 4.51E−07 |
| NADH to ubiquinone | | | |
| Respiratory electron transport | Down | 105 | 2.74E−06 |
| chain | | | |
| Regulation of prostaglandin | Up | 11 | 3.67E−06 |
| secretion | | | |
| Mitochondrial respiratory chain | Down | 94 | 1.39E−05 |
| complex assembly | | | |
| Prostaglandin secretion | Up | 13 | 1.03E−05 |

TABLE 5
Changes in signaling pathways using RNA seq data from
brown adipocytes exposed to vehicle or LH treatment.

| | | Number of | |
|---|---|---|---|
| Signaling pathway | Direction | genes | P value |
| Positive regulation of | Up | 18 | 0.00016 |
| fatty acid transport | | | |
| Response to cold | Down | 44 | 0.00022 |
| Response to fatty acid | Down | 51 | 0.00048 |

### Conclusion

This study shows that LHCGR is expressed in both mature brown and white adipocytes at a relatively high expression level. The presence of the receptor suggests a direct effect of LH and hCG, which was proven in both white and brown cells where LH influenced gene expression of more than 400 genes. High LH suppressed mitochondrial function that alone may be important and prevention of this could increase longevity and function of the cells and may ultimately increase the risk of age-related diseases such as metabolic syndrome, thrombosis and dementia. Moreover, inhibition of aerobe and anaerobe metabolism electron transport, NADH synthesis, Krebs cycle, fatty acid metabolism, thyroid hormone metabolism, prostaglandins highlight a central role in regulation of adipocyte function and fat accumulation. Interestingly, there was a pronounced effect on cold-induced thermogenesis, which was suppressed in both white and brown fat cells when exposed to high LH, which is in line with the shown suppression of UCP1 that is central for heat generation as is DIO2 responsible for thyroid hormone conversion into T3 that also influence temperature and obesity.

In sum, the data presented here indicates that white and brown cell adipocyte function may be improved by treatment with an LH or LHCGR blocking antibody.

### Example 10—Blocking LH Action Using LH-β Ab or LHCGR Ab or with a GnRH Agonist/Antagonist Reduce Calcium Excretion Both in Normal Male Mice but Also in a Menopause Mouse Model

### Aim of Study

The aim of this study was to validate that LH is responsible for the high calcium excretion observed after injection of LH in mice by analyzing the effects on calcium excretion and kidney size in normal male mice and bilateral ovariectomized female mice treated with IgG2b (control) or 1 of 3 test articles (LH-β ab, LHCGR ab or GnRH antagonist).

### Materials and Methods

### Study Group

Bilateral ovariectomized female C57BL/6JRj mice, 8 weeks of age, were included in the study and administered with IgG2b (control) or 1 of 3 test articles (LH-β ab, LHCGR ab or GnRH antagonist) either at study day 1, study day 1 and 5 or once a day for 9 days. The route of administration was through intraperitoneal (i.p.) injection. The study included be 6 groups in total (Table 6); group 1 (female, n=6) was treated i.p. with IgG2b (control) on study day 1 and day 5, group 2 (female, n=3) was treated i.p. with LH-β ab at study day 1, group 3 (female, n=1) was treated i.p. with LH-β AB on study day 1 and day 5, Group 4 (female, n=3) was treated i.p. with LHCGR ab at study day 1, group 5 (female, n=1) was treated i.p. with LHCGR ab on study day 1 and day 5, group 6 (female, n=3) was treated i.p. with GnRH antagonist once a day (q.d.) for 9 days. Blood for preparation of serum was collected prior to dosing on study day 1 and at termination on study day 10. Urine and kidneys were collected on day 10.

### Study Animals

Seventeen (17) bilateral ovariectomized C57BL/6JRj female mice 8 weeks of age, were supplied by Janvier Labs France. The ovaries from the female mice were surgically removed by the supplier (see also Table 6). Fourteen (14) C57BL/6JRj male mice were also stratified into the different groups except that no male mice were treated with GnRH antagonist, and the male mice were therefore only stratified into groups 1-5 (see also Table 7).

TABLE 6
Schematic illustration of the six (female mice) treatment groups

| | Mice | | Conc. | Dose | Dose volume |
|---|---|---|---|---|---|
| Group | (n) | Treatment | (mg/mL) | (mg) | (mL) |
| 1 | 6 | Control (IgG2b) | 0.34 | 0.17 | 0.5 |
| 2 | 3 | LH-β ab | 0.34 | 0.17 | 0.5 |
| 3 | 1 | LH-β ab | 0.34 | 0.17 × 2 | 0.5 |
| 4 | 3 | LHCGR ab | 0.34 | 0.17 | 0.5 |
| 5 | 1 | LHCGR ab | 0.34 | 0.17 × 2 | 0.5 |
| 6 | 3 | GnRH antagonist | 0.02 | 0.5 ug/g | 25 mL/kg |

TABLE 7
Schematic illustration of the five (male mice) treatment groups

| | Mice | | Conc. | Dose | Dose volume |
|---|---|---|---|---|---|
| Group | (n) | Treatment | (mg/mL) | (mg) | (mL) |
| 1 | 6 | Control (IgG2b) | 0.34 | 0.17 | 0.5 |
| 2 | 3 | LH-β ab | 0.34 | 0.17 | 0.5 |
| 3 | 1 | LH-β ab | 0.34 | 0.17 × 2 | 0.5 |
| 4 | 3 | LHCGR ab | 0.34 | 0.17 | 0.5 |
| 5 | 1 | LHCGR ab | 0.34 | 0.17 × 2 | 0.5 |

### Antibodies

See example 8.

### GnrH Antagonist

The GnRH antagonist Cetrorelix (Merck) was used in the present example.

### Results

Injection of antibodies targeting LH-β or LHCGR changed kidney weight and calcium in male mice and in a mouse model of female menopause (bilateral ovariectomized C57BL/6JRj female mice) (FIG. 14).

The kidney weight was slightly lower in mice treated with antibodies targeting LH-β or LHCGR compared with the vehicle treatments in both male mice and in female mice without ovaries (FIG. 14A).

The kidney weight was also slightly lower in mice treated with GnrH antagonist suggesting that all treatments lowering LH activity had lower body weight after 10 days of treatment compared with vehicle treatment (IgG2b). Interestingly, LH and LHCGR antibodies and GnRH antagonist suppressed calcium excretion in the female mouse model of osteoporosis (FIG. 14B). The effect was pronounced, and a high dosage of LH-β ab suppressed calcium excretion with 70% while the average effect was a 35% decrease in calcium excretion following any treatment compared with vehicle. The same phenomenon was observed in male mice where all concentrations of both antibodies targeting LH-β and LHCGR reduced calcium excretion (FIG. 14C).

### Conclusion

This study suggests that blocking LH action either by blocking LH-β or the receptor LHCGR with antibodies or using a GnRH agonist or antagonist will reduce calcium excretion both in normal male mice but also in a model of menopause. This finding is intriguing because it demonstrates that the observed increase in calcium excretion following LH treatment of male mice is opposite to the function of antibodies blocking LH-β or LH receptor action (LHCGR). Moreover, these data can be extrapolated into female mice without ovaries, which is a good model of menopause, since the effect is similar of both antibodies and GnRH antagonist blocking LH production or activity. This shows that LH is an important regulator of calcium excretion in both males and females. Moreover, LH increases kidney size, but this study shows that antibodies targeting LH-β or LHCGR reduce kidney size suggesting opposite actions on the renal function.

The conclusion of the study is therefore that blocking circulating high levels of LH in menopause diminishes urinary calcium loss, which will reduce bone resorption and the risk of osteoporosis. Further, reduction in calcium excretion is also obtainable in normal male mice.

Sequence list

| SEQ | | |
|---|---|---|
| ID NO: | Name* | Sequence |
| 1 | LH beta amino acid | See sequence listing |
| 2 | LHCGR amino acid | See sequence listing |
| 3 | LH ab VL CDR1 | QNIVQSNGNTY |
| | LH ab VL CDR2 | KVS |
| 4 | LH ab VL CDR3 | FQGSHVPLT |
| 5 | LH ab VL full length | See sequence listing |
| 6 | LH ab VH CDR1 | GYTFTDYV |
| 7 | LH ab VH CDR2 | IYPGSGST |
| 8 | LH ab VH CDR3 | AHLYYADYDFSY |
| 9 | LH ab VH full length | See sequence listing |

*LH is an abbreviation of luteinizing hormone; LHCGR is an abbreviation of luteinizing hormone/choriogonadotropin receptor; ab is an abbreviation of antibody; VL is an abbreviation of variable light chain; VH is an abbreviation of variable heavy chain.

### REFERENCES

Eastell R., et al.: Postmenopausal osteoporosis. Nature Reviews Disease Primers, 2016, 2 16069. (doi: 10.1038/nrdp.2016.69)
Ebeling P R, et. al.: Bone turnover markers and bone density across the menopausal transition. The Journal of Clinical Endocrinology & Metabolism, 1996, 81 3366-3371. (doi: 10.1210/jcem.81.9.8784098)
Odén A, et al.: Assessing the Impact of Osteoporosis on the Burden of Hip Fractures. Calcified Tissue International, 2013, 92 42-49. (doi: 10.1007/s00223-012-9666-6)·
Sidebottom A C., et al.: Pregnancy-related changes in body fat, European Journal of Obstetrics & Gynecology and Reproductive Biology, 2001, 94 216-223. (doi: 10.1016/S0301-2115 (00) 00329-8)·
Gertner, J. M. et al.: Pregnancy as state of physiologic absorptive hypercalciuria. Am. J. Med. 1986, 81, 451-6. (doi: 10.1016/0002-9343 (86) 90298-6).
Payne, R. B. et al.: Albumin-adjusted calcium concentration in serum increases during normal pregnancy. Clin. Chem. 1990, 36, 142-4.
Cross, N. A. et al.: Calcium homeostasis and bone metabolism during pregnancy, lactation, and postweaning: A longitudinal study. Am. J. Clin. Nutr. 1995, 61, 514-523. (doi: 10.1093/ajcn/61.3.514).
Sun, L. et al.: FSH Directly Regulates Bone Mass. Cell 2006, 125, 247-260. (doi: 10.1016/j.cell.2006.01.051).
Liu, P. et al.: Blocking FSH induces thermogenic adipose tissue and reduces body fat. Nature 2017. (doi: 10.1038/nature22342)

1.-35. (canceled)

36. A method for treating or alleviating a disease or disorder in a subject in need thereof, the method comprising administering to the subject one or more of
i) an antibody;
ii) a small molecule targeting luteinizing hormone (LH);
iii) a luteinizing hormone choriogonadotropin receptor (LHCGR); and,
iv) a GnRH agonist or GnRH antagonist;
at an amount effective for one or more of:
i) reduction of fat mass and, or inhibition of increase in fat mass in a female subject who is at least one of perimenopausal, menopausal and postmenopausal;
ii) treatment and, or alleviation of osteoporosis symptoms in a perimenopausal or postmenopausal female subject;
iii) treatment and, or alleviation of osteoporosis symptoms in a male subject;
iv) treatment and, or alleviation of hypergonadotropic hypogonadism in a male subject;
v) treatment and, or alleviation of symptoms of perimenopause, menopause and/or postmenopause;
vi) treatment and, or alleviation of suppressed mitochondrial function; and
vii) treatment and, or alleviation of risk of age-related diseases.

37. The method according to claim 1, wherein said antibody or small molecule binds specifically to an epitope in the β-subunit of LH.

38. The method according to claim 1, wherein said antibody or small molecule binds specifically to an epitope in the β-subunit of LH and blocks activation and/or binding of luteinizing hormone (LH) to luteinizing hormone/choriogonadotropin receptor (LHCGR).

39. The method according to claim 1, wherein said antibody and/or small molecule binds specifically to an epitope in the β-subunit of LH, within position 21-141 of SEQ ID NO: 1.

40. The method according to claim 1, wherein at least one of
i) an antibody targeting luteinizing hormone (LH);
ii) a luteinizing hormone/choriogonadotropin receptor (LHCGR); and,
iii) a GnRH agonist or GnRH antagonist; is administered.

41. The method according to claim 1, wherein an antibody targeting luteinizing hormone (LH) is administered.

42. The method according to claim 1, wherein an antibody targeting LHCGR is administered.

43. The method according to claim 1, wherein said antibody and/or small molecule binds specifically to an epitope on LHCGR and blocks at least one of activation and binding of luteinizing hormone (LH) to luteinizing hormone/choriogonadotropin receptor (LHCGR).

44. The method according to claim 1, wherein said antibody and, or small molecule binds specifically to an epitope on LHCGR, within position 27-362 of SEQ ID NO: 2.

45. The method according to claim 1, wherein the antibody is selected from the group consisting of a polyclonal antibody, a monoclonal antibody, an antibody wherein the heavy chain and the light chain are connected by a flexible linker, an Fv molecule, an antigen binding fragment, a Fab fragment, a Fab′ fragment, a F(ab′)2 molecule, a fully human antibody, a humanized antibody, a chimeric antibody, a fragment of an antibody and a single-domain antibody (sdAb) (nanobody).

46. The method according to claim 1, wherein a small molecule targeting luteinizing hormone (LH) and/or luteinizing hormone/choriogonadotropin receptor (LHCGR); and/or being a GnRH agonist or GnRH antagonist is administered.

47. The method according to claim 1, wherein said small molecule is selected from the group consisting of one or more of Goserelin (CAS No 65807-02-5), Leuprorelin (CAS No 53714-56-0), Triptorelin (CAS No 57773-63-4), and Nafarelin (CAS No 76932-56-4).

48. The method according to claim 1, wherein said subject has a level of one or more of:
i) luteinizing hormone (LH) above 10 IU/L in blood serum;
ii) anti mullerian hormone (AMH) below 10 pmol/L in blood serum; and
iii) estrogen below 30 pg/mL in blood serum.

49. The method according to claim 1, wherein said male subject has undergone hemi-orchiectomy.

50. The method according to claim 1, wherein the reduction of fat mass and, or of increase in fat mass, is reduction of visceral and/or subcutaneous fat mass and, or inhibition of increase in visceral and subcutaneous fat mass.

51. The method according to claim 1, wherein said subject has a BMI in the range 18.5-24.9.

52. The method according to claim 1, wherein said subject has a BMI in the range 25-29.9.

53. The method according to claim 1, for reducing fat mass and, or inhibiting an increase in fat mass, wherein said subject is an obese perimenopausal, menopausal or postmenopausal female.

54. The method according to claim 1, for reducing fat mass and, or inhibiting an increase in fat mass is in an obese perimenopausal, menopausal, or postmenopausal female subject having a BMI of 30 or higher.

55. The method according to claim 1, wherein
i) the symptoms or manifestations of one or more of perimenopause, menopause, or postmenopause comprise one or more of hot flashes, night sweats, osteoporosis, weight gain, high cortisol, hypothyroidism, conversion of T4 to T3, and primary hyperparathyroidism are alleviated; and, or
ii) risk of age-related diseases selected from the group consisting of metabolic syndrome, thrombosis, dementia and Alzheimer's disease is reduced.


SEQUENCE LISTING (9 sequences)

SEQ ID NO: 1 - protein, 141 aa, synthetic construct
MERLQGLLLWLLLSPSVVWASRGPLRPLCRPVNATLAAENEFCPVCITFTTSICAGYCPSMVRVLPAALPPVPQPVCTYRELAFASVRLPGCPPGVDPIVSFPVALSCRCGPCRLSSSDCGGPRTQPMACDLPHLPGLLLL

SEQ ID NO: 2 - protein, 700 aa, synthetic construct
MGRRVPALRQLLVLAMLVLKQSQLHSPELSGSRCPEPCDCAPDGALRCPGPRAGLARLSLTYLPVKVIPSQAFRGLNEVVKIEISQSDSLERIEANAFDNLLNLSEILIQNTKNLLYIEPGAFTNLPRLKYLSICNTGIRTLPDVSKISSSEFNFILEICDNLYITTIPGNAFQGMNNESITLKLYGNGFEEVQSHAFNGTTLISLELKENIYLEKMHSGTFQGATGPSILDVSSTKLQALPSHGLESIQTLIATSSYSLKTLPSREKFTSLLVATLTYPSHCCAFRNLPKKEQNFSFSIFENFSKQCESTVREANNETLYSAIFEENELSGWDYDYDFCSPKTLQCTPEPDAFNPCEDIMGYAFLRVLIWLINILAIFGNLTVLFVLLTSRYKLTVPRFLMCNLSFADFCMGLYLLLIASVDSQTKGQYYNHAIDWQTGSGCSAAGFFTVFASELSVYTLTVITLERWHTITYAVQLDQKLRLRHAIPIMLGGWIFSTLMATLPLVGVSSYMKVSICLPMDVESTLSQVYILSILLLNAVAFVVICACYVRIYFAVQNPELTAPNKDTKIAKKMAILIFTDFTCMAPISFFAISAAFKVPLITVTNSKVLLVLFYPVNSCANPFLYAVFTKAFQRDFFLLLSRFGCCKHRAELYRRKEFSACTFNSKNGFPRSSKPSQAALKLSIVHCQQPTPPRVLIQ

SEQ ID NO: 3 - protein, 11 aa, synthetic construct
QNIVQSNGNTY

SEQ ID NO: 4 - protein, 9 aa, synthetic construct
FQGSHVPLT

SEQ ID NO: 5 - protein, 202 aa, synthetic construct
MKLPVRLLVLMFWIPVSSSDVLMTQIPLSLVVSLGDQASISCRSSQNIVQSNGNTYLEWYLQKPGQSPKLLIYKVSNRFSGVPDRFSGSGSGTDFTLKISRVEAEDLGVYYCFQGSHVPLTFGAGTKLELKRADAAPTVSIFPPSSEQLTSGGASVVCFLNNFYPKDINVKWKIDGSERQNGVLNSWTDQDSKDSTYSMSST

SEQ ID NO: 6 - protein, 8 aa, synthetic construct
GYTFTDYV

SEQ ID NO: 7 - protein, 8 aa, synthetic construct
IYPGSGST

SEQ ID NO: 8 - protein, 12 aa, synthetic construct
AHLYYADYDFSY

SEQ ID NO: 9 - protein, 235 aa, synthetic construct
MEWRIFLFILSGTAGVHSQVQLQQSGPELVKPGTSLKLSCKASGYTFTDYVLTWVKQRTGQGLEWIGEIYPGSGSTSYNEKFRGKATLTADKSSNTAYMQLSSLTSEDSAVYFCAHLYYADYDFSYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLE